(19) United States
(12) Patent Application Publication
(10) Pub. No.: US 2010/0267744 A1
(43) Pub. Date: Oct. 21, 2010

(54) Pharmaceutical Compositions

(75) Inventors: Kevin John Bittorf, Boston, MA (US); Jeffrey P. Katstra, Andover, MA (US); Filipe Gaspar, Oeiras (PT)

Correspondence Address: Jonathan P. O''Brien, Ph.D.;Honigman Miller Schwartz & Cohn LLP, 350 East Michigan Avenue, Suite 300, Kalamazoo, MI, 49007, US

(73) Assignee: Vertex Pharmaceuticals Incorporated, Cambridge, MA (US)

(21) Appl. No.: 12/767,409

(22) Filed: Apr. 26, 2010

Related U.S. Application Data
(63) Continuation of application No. 11/687,716, filed on Mar. 19, 2007, now abandoned.
(60) Provisional application No. 60/784,428, filed on Mar. 20, 2006.

(51) Int. Cl. A61K 31/497; A61P 31/12

(52) U.S. Cl. 514/255.05

(57) ABSTRACT

Forms and formulations of VX-950 and uses thereof.

### CROSS-REFERENCE TO RELATED APPLICATIONS

This application claims priority to U.S. Application Ser. No. 60/784,428, filed on Mar. 20, 2006. The disclosure of the prior application is considered part of (and is incorporated by reference in) the disclosure of this application.

### TECHNICAL FIELD

This disclosure relates to pharmaceutical compositions.

### BACKGROUND

Infection by hepatitis C virus (“HCV”) is a compelling human medical problem. HCV is recognized as the causative agent for most cases of non-A, non-B hepatitis, with an estimated human sero-prevalence of 3% globally [A. Alberti et al., “Natural History of Hepatitis C,” J. Hepatology, 31., (Suppl. 1), pp. 17-24 (1999)]. Nearly four million individuals may be infected in the United States alone [M. J. Alter et al., “The Epidemiology of Viral Hepatitis in the United States, Gastroenterol. Clin. North Am., 23, pp. 437-455 (1994); M. J. Alter “Hepatitis C Virus Infection in the United States,” J. Hepatology, 31., (Suppl. 1), pp. 88-91 (1999)].

Upon first exposure to HCV, only about 20% of infected individuals develop acute clinical hepatitis while others appear to resolve the infection spontaneously. In almost 70% of instances, however, the virus establishes a chronic infection that persists for decades [S. Iwarson, “The Natural Course of Chronic Hepatitis,” FEMS Microbiology Reviews, 14, pp. 201-204 (1994); D. Lavanchy, “Global Surveillance and Control of Hepatitis C,” J. Viral Hepatitis, 6, pp. 35-47 (1999)]. This usually results in recurrent and progressively worsening liver inflammation, which often leads to more severe disease states such as cirrhosis and hepatocellular carcinoma [M. C. Kew, “Hepatitis C and Hepatocellular Carcinoma”, FEMS Microbiology Reviews, 14, pp. 211-220 (1994); I. Saito et. al., “Hepatitis C Virus Infection is Associated with the Development of Hepatocellular Carcinoma,” Proc. Natl. Acad. Sci. USA, 87, pp. 6547-6549 (1990)]. It is estimated that HCV infects 170 million persons worldwide. Over the next ten years, as a larger proportion of patients who are currently infected enter the third decade of their infection, the number of deaths attributed to hepatitis C is expected to significantly increase. Unfortunately, there are no broadly effective treatments for the debilitating progression of chronic HCV.

There are not currently any fully-satisfactory anti-HCV agents or treatments. Interferon, as well as pegylated Interferon, is used to treat HCV, and can also be dosed in combination with Ribavirin. Any treatment regimen containing Interferon is known to have significant side effects, and there is thus a significant unmet medical need for a safe, effective, oral therapy to treat HCV. Moreover, the prospects for effective anti-HCV vaccines remain uncertain.

VX-950 is a competitive, reversible peptidomimetic HCV NS3/4A protease inhibitor with a steady state binding constant (ki*) of 3 nM (and with a Ki of 8 nM) [WO 02/018369].

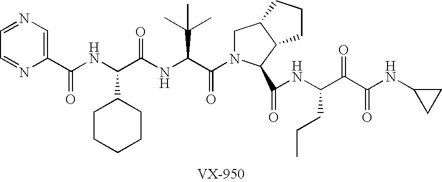

VX-950 is highly insoluble in water.

### SUMMARY

The inventors have discovered improved forms and formulations of VX-950, e.g., those having improved bioavailability relative to crystalline VX-950. These forms and formulations are useful for treating HCV infection. It has also been discovered that the presence of two or more polymers (e.g., a plurality of polymers) in formulations containing VX-950 can help convey improved properties, e.g., can stabilize the VX-950.

Accordingly, in one aspect, the disclosure features a preparation of amorphous VX-950, for example a preparation of VX-950 that is substantially pure of impurities and/or crystalline VX-950. For example, in one embodiment, the disclosure features formulations containing VX-950 in the amorphous form, which enhances the metastable solubility of VX-950 relative to the crystalline form, and thus provides improved bioavailability. The disclosure includes a number of formulations which contain VX-950 in the amorphous form.

In one aspect, the disclosure features a solid (e.g. spray dried) dispersion comprising amorphous VX-950 and a plurality of polymers. The dispersion can include, e.g., less than about 40% (less than about 30%, less than about 20%, less than about 15%, less than about 10%, less than about 5%, less than about 1%) of crystalline VX-950, e.g., be substantially free of crystalline VX-950.

In a preferred embodiment, the solid dispersion exhibits a predetermined level of physical and/or chemical stability. E.g., the solid dispersion retains about 50%, about 60%, about 70%, about 80%, about 90%, about 95%, about 98%, or about 99%, of amorphous VX-950 when stored at 25° C. in a closed water tight container, e.g., an amber glass vial or high density polyethylene (HDPE) container.

In a preferred embodiment, the solid dispersion exhibits at least about 50%, at least about 60%, at least about 70%, at least about 80%, at least about 90%, at least about 95%, at least about 98%, or at least about 99% of the bioavailability when administered to a fed subject as when administered to a fasted subject.

In a preferred embodiment, the solid dispersion exhibits at least about 50%, at least about 60%, at least about 70%, at least about 80%, at least about 90%, at least about 95%, at least about 98%, or at least about 99% of the bioavailability when administered to a fasted subject as when administered to a fed subject.

In some embodiments, the solid dispersion also includes a surfactant (e.g., sodium lauryl sulfate (SLS) or vitamin E or a derivative thereof) or inert pharmaceutically acceptable substance. In some embodiments, the surfactant is SLS. In some embodiments, the surfactant is vitamin E or a derivative thereof (e.g., vitamin E TPGS).

In some embodiments, the surfactant is present in an amount of between about 0.1% and about 10% (e.g., up to about 5%, up to about 4%, up to about 3%, up to about 2%, at about 1%).

In some embodiments, the plurality of polymers includes two polymers (e.g., one or more than one water-soluble polymer or partially water-soluble polymer). In some embodiments, the plurality of polymers includes a cellulose polymer.

In some embodiments, the cellulose polymer is hydroxypropylmethylcellulose (HPMC; “hypromellose”) or hydroxypropylmethylcellulose acetate succinate (HPMCAS).

In some embodiments, the plurality of polymers includes two cellulose polymers, e.g., one of the two cellulose polymers is hydroxypropylmethylcellulose (HPMC), and/or one of the two cellulose polymers is hydroxypropylmethylcellulose acetate succinate (HPMCAS). In some embodiments, the solid dispersion includes HPMC and HPMCAS.

In some embodiments, the solid dispersion further includes a surfactant, a mixture of polymers, or an inert pharmaceutically acceptable substance. For example, the solid dispersion can include a mixture of polymers, and the a mixture of polymers can include one or more than one water-soluble polymer or partially water-soluble polymer, e.g., a combination of the polymers described herein.

In some embodiments, the dispersion includes a surfactant or inert pharmaceutically acceptable substance. For example, the surfactant is SLS or vitamin E or a derivative thereof (e.g., vitamin E TPGS). In some embodiments, the surfactant is present in an amount of between about 0.1% and about 10% (e.g., up to about 5%, up to about 4%, up to about 3%, up to about 2%, at about 1%). The amount of surfactant present in the solid dispersion is dependent on a variety of factors, including, for example, the chemical nature of the surfactant. In some embodiments, the surfactant is present in an amount from about 0.1 to about 15%, for example from about 0.1% to about 5%, preferably about 1% by weight in the solid dispersion.

In some embodiments, the VX-950 has improved physical or chemical stability relative to amorphous VX-950 without the presence of a mixture of polymers. In some embodiments the solid dispersion has a higher glass transition temperature than the glass transition temperature of neat amorphous VX-950. In some embodiments, the VX-950 has a relaxation rate that is lower than the relaxation rate of neat amorphous VX-950.

In some embodiments, the solid dispersion includes a mixture of polymers that includes a cellulosic polymer, for example an HPMC polymer or an HPMCAS polymer.

In some embodiments, the mixture of polymers (e.g., HPMC and HPMCAS) is present in the solid dispersion in an amount of from about 10% by weight to about 80%, for example from about 30% to about 75%, for example, about 70%, about 50%, or about 49.5% by weight.

In some embodiments, VX-950 is present in the solid dispersion in an amount of from about 10% by weight to about 80% by weight, for example from about 30% to about 75%, for example, about 70%, about 50%, or about 49.5% by weight. In some embodiments, VX-950 is present in the solid dispersion in an amount of greater than about 80% (e.g., about 90%).

In some embodiments, the solid dispersion includes a surfactant, for example, sodium lauryl sulfate or vitamin E or a derivative thereof (e.g., vitamin E TPGS).

In some embodiments, substantially all of the VX-950 is present in the solid dispersion in amorphous form.

In some embodiments, the VX-950 is a mixture of the L-isomer and the D-isomer.

In some embodiments, the VX-950 is substantially pure L-isomer.

In some embodiments, the solid dispersion is obtained by spray drying.

In some embodiments, the plurality of polymers decreases the amount or rate of crystallization of the amorphous VX-950 by at least about 10% (e.g., by at least about 20%, by at least about 30%, by at least about 40%, by at least about 50%, by at least about 60%, by at least about 70%, by at least about 80%, or by at least about 90%) compared to a solid dispersion without being in the presence of the plurality of polymers.

In some embodiments, the plurality of polymers improves the physical stability of the amorphous VX-950 by at least about 10% (e.g., by at least about 20%, by at least about 30%, by at least about 40%, by at least about 50%, by at least about 60%, by at least about 70%, by at least about 80%, or by at least about 90%) compared to a solid amorphous dispersion without being in the presence of the plurality of polymers, or as compared to neat VX-950 (e.g., without polymers).

In some embodiments, the plurality of polymers increases the chemical or physical stability (e.g., as measured by X ray powder dispersion) of the solid dispersion when stored (e.g., at 2-8° C., e.g. 4° C. or at room temperature) by at least about 10% (e.g., by at least about 20%, by at least about 30%, by at least about 40%, by at least about 50%, by at least about 60%, by at least about 70%, by at least about 80%, or by at least about 90%) compared to a solid dispersion without being in the presence of the plurality of polymers.

In some embodiments, the VX-950 has improved physical or chemical stability (e.g., in gastric fluids, SGF, intestinal fluids, SIF) relative to amorphous VX-950 without being in the presence of the plurality of polymers. The plurality of polymers can affect enteric pH dissolution along the digestive tract.

In some embodiments, the plurality of polymers (e.g., HPMC and HPMCAS) is present in an amount of from about 5% by weight to about 80% by weight (e.g., from about 10% to about 70%, from about 20% to about 60%, from about 30% to about 50% by weight).

In a preferred embodiment, the solid dispersion includes about 45% to about 85% VX-950, about 5% to about 25% of an HPMC polymer, such as HPMC60SH50 or HPMC-E50, about 5% to about 30% of an HPMCAS polymer, such as HPMCAS-HG, and about 0.1% to about 10% of a surfactant, such as SLS or vitamin E or a derivative thereof (e.g., vitamin E TPGS), wherein the HPMC and HPMCAS together account for about 90%, about 95%, about 98%, about 99%, or about 100% of the total polymer present.

In a preferred embodiment, the solid dispersion exhibits a predetermined level of physical and/or chemical stability. E.g., the solid dispersion retains about 50%, about 60%, about 70%, about 80%, about 90%, about 95%, about 98%, or about 99%, of amorphous VX-950 when stored at 25° C. in a closed water tight container, e.g., an amber glass vial or high density polyethylene (HDPE) container.

In some embodiments, the solid dispersion includes between about 50% and about 60% (e.g., about 55%) VX-950, between about 15% and about 25% (e.g., about 19.6%) of an HPMC polymer, such as HPMC60SH50, between about 20% and about 30% (e.g., about 24.4%) of an HPMCAS polymer, such as HPMCAS-HG, and between about 0.1% and about 5% (e.g., about 1%) of a surfactant, such as SLS.

In some embodiments, the solid dispersion includes solid dispersion including between about 50% and about 60% (e.g., about 55%) VX-950, between about 25% and about 35% (e.g., about 29.3%) of an HPMC polymer, such as HPMC60SH50, between about 10% and about 20% (e.g., about 14.7%) of an HPMCAS polymer, such as HPMCAS-HG, and between about 0.1% and about 5% (e.g., about 1%) of a surfactant, such as SLS.

In some embodiments, the solid dispersion includes between about 55% and about 65% (e.g., about 60%) VX-950, between about 10% and about 20% (e.g., about 14.6%) of an HPMC polymer, such as HPMC60SH50, between about 20% and about 30% (e.g, about 24.4%) of an HPMCAS polymer, such as HPMCAS-HG, and between about 0.1% and about 5% (e.g., about 1%) of a surfactant, such as SLS.

In some embodiments, the solid dispersion includes between about 60% and about 70% (e.g., about 65%) VX-950, between about 12% and about 22% (e.g., about 17%) of an HPMC polymer, such as HPMC60SH50, between about 12% and about 22% (e.g., about 17%) of an HPMCAS polymer, such as HPMCAS-HG, and between about 0.1% and about 5% (e.g., about 1%) of a surfactant, such as SLS.

In some embodiments, the solid dispersion includes between about 65% and about 75% (e.g., about 70%) VX-950, between about 15% and about 25% (e.g., about 19.3%) of an HPMC polymer, such as HPMC60SH50, between about 5% and about 15% (e.g., about 9.7%) of an HPMCAS polymer, such as HPMCAS-HG, and between about 0.1% and about 5% (e.g., about 1%) of a surfactant, such as SLS.

In some embodiments, a first polymer is present in an amount of between about 1% and about 99% and a second polymer is present in an amount of between about 1% and 99%, wherein the amounts of the first and second polymers amount to 100% of the total polymer present in the solid dispersion.

In some embodiments, the first polymer is present in an amount of between about 28% and about 38% (e.g., about 33%) and the second polymer is present in an amount of between about 62% and about 72% (e.g., about 67%) of the amount of total polymer.

In some embodiments, the first polymer is present in an amount of between about 47% and about 57% (e.g., about 52%) and the second polymer is present in an amount of between about 43% and about 53% (e.g., about 48%) of the amount of total polymer.

In some embodiments, the first polymer is present in an amount of between about 58% and about 68% (e.g., about 63%) and the second polymer is present in an amount of between about 32% and about 42% (e.g., about 37%) of the amount of total polymer.

In some embodiments, the first polymer is present in an amount of between about 45% and about 55% (e.g., about 50%) and the second polymer is present in an amount of between about 45% and about 55% (e.g., about 50%) of the amount of total polymer.

In some embodiments, the first polymer is HPMCAS. In some embodiments, the second polymer is HPMC. In some embodiments, the first polymer is HPMC and the second polymer is HPMCAS.

In one embodiment, the disclosure provides a solid dispersion of VX-950, such as an amorphous solid dispersion. For example an amorphous solid dispersion including VX-950, a mixture of polymers, and optionally one or more solubility enhancing surfactant is provided. The dispersion can enhance the aqueous solubility and bioavailability of VX-950 upon oral dosing of the solid dispersion to a mammal (e.g., a rat, dog or human). In certain aspects, at least a portion of the VX-950 in the solid dispersion is in the amorphous state (e.g., at least about 50%, at least about 55%, at least about 60%, at least about 65%, at least about 70%, at least about 75%, at least about 80%, at least about 85%, at least about 90%, at least about 95%, at least about 98%, or at least about 99%). In preferred embodiments, the solid dispersion is essentially or substantially free of crystalline VX-950.

In certain solid dispersions, VX-950 (e.g., amorphous VX-950) is present in an amount of up to about 99%, for example up to about 98%, up to about 95%, up to about 90%, up to about 85%, up to about 80%, up to about 70%, preferably up to about 70%, up to about 65%, up to about 60%, up to about 55%, and more preferably up to about 50% of the total weight of the solid dispersions. In other embodiments, VX-950 is present in an amount of at least about 1% of the solid dispersion, for example at least about 2%, at least about 3%, at least about 4%, preferably at least about 5%, at least about 6%, at least about 7%, at least about 8%, at least about 9%, more preferably at least about 10%, and even more preferably at least about 50%. As shown in the examples herein, a solid dispersion, wherein the VX-950 is present in an amount of about 50% by weight (and more specifically about 49.5%) is included within this disclosure.

In some embodiments, when VX-950 is in a solid dispersion, at least about 60% by weight of the VX-950 is in an amorphous form, for example, at least about 65%, at least about 70%, at least about 75%, preferably at least about 80%, at least about 85%, at least about 90%, at least about 95%, at least about 98%, or at least about 99%. Dispersions wherein all or substantially all the VX-950 is in amorphous form, are also included.

In some embodiments, a dispersion including VX-950 includes a mixture of the L-isomer and the D-isomer (e.g., 1:1) of VX-950, or VX-950 may be in a substantially pure form of either isomer. For example, mixtures of about 60:40 of L:D (+/−5%) are included. In certain embodiments, the VX-950 is in an amount of about 95%, about 98%, or greater than about 98% of the L-isomer.

An amorphous solid dispersion generally exhibits a glass transition temperature, where the dispersion makes a transition from a glassy solid to a rubbery composition. In general, the higher the glass transition temperature, the greater the physical stability of the dispersion. The existence of a glass transition temperature generally indicates that at least a large portion of the composition (e.g., dispersion) is in an amorphous state. The glass transition temperature (T_{g}) of a solid dispersion suitable for pharmaceutical applications is generally at least about 50° C. In some embodiments, higher temperatures are preferred. Therefore, in some embodiments, a solid dispersion of this disclosure has a T_{g} of at least about 100° C. (e.g., at least about 100° C., at least about 105° C., at least about 110° C., at least about 115° C., at least about 120° C., at least about 125° C., at least about 130° C., at least about 135° C., at least about 140° C., at least about 150° C., at least about 160° C., at least about 170° C., at least about 175° C., at least about 180° C., or at least about 190° C.). In some preferred embodiments, the T_{g} is up to about 200° C. Unless otherwise noted, the glass transition temperatures described herein are measured under dry conditions.

In another aspect, the disclosure features pharmaceutical compositions of amorphous VX-950 and a plurality of polymers, e.g., as described herein.

In a preferred embodiment, the solid dispersion exhibits a predetermined level of physical and/or chemical stability. E.g., the solid dispersion retains about 50%, about 60%, about 70%, about 80%, about 90%, about 95%, about 98%, or about 99%, of amorphous VX-950 when stored at 25° C. in a closed water tight container, e.g., an amber glass vial or high density polyethylene (HDPE) container.

In some embodiments, the amorphous VX-950 is substantially free of crystalline VX-950.

In some embodiments, a pharmaceutical composition includes an amorphous VX-950 and a plurality of polymers as a solid dispersion, and one or more of a surfactant, inert pharmaceutically acceptable substance, or pharmaceutically acceptable carrier.

In some embodiments, the plurality of polymers includes one or more than one water-soluble polymer or partially water-soluble polymer.

In some embodiments, the VX-950 has improved physical or chemical stability relative to crystalline VX-950.

In some embodiments, the plurality of polymers decreases the amount or rate of crystallization of the amorphous VX-950 by at least about 10% (e.g., by at least about 20%, by at least about 30%, by at least about 40%, by at least about 50%, by at least about 60%, by at least about 70%, by at least about 80%, or by at least about 90%) compared to a pharmaceutical composition without being in the presence of the plurality of polymers, or as compared to neat VX-950.

In some embodiments, the plurality of polymers increases the chemical or physical stability of the pharmaceutical composition by at least about 10% (e.g., by at least about 20%, by at least about 30%, by at least about 40%, by at least about 50%, by at least about 60%, by at least about 70%, by at least about 80%, or by at least about 90%) compared to a pharmaceutical composition without being in the presence of the plurality of polymers, or as compared to neat VX-950.

In some embodiments, the VX-950 has improved physical or chemical stability relative to amorphous VX-950 without being in the presence of the plurality of polymers.

In some embodiments, the plurality of polymers includes HPMC or HPMCAS.

In some aspects, the disclosure features pharmaceutical composition that includes:

an amorphous solid dispersion of VX-950, wherein said VX-950 makes up about 25-85% wt/wt of the pharmaceutical composition,

a plurality of polymers, wherein the plurality includes two cellulose polymers, and wherein the plurality of polymers makes up about 15-75% wt/wt of the pharmaceutical composition, and

a surfactant, wherein said surfactant makes up about 0.5-2% wt/wt of the pharmaceutical composition.

In some embodiments, wherein a cellulose polymer is HPMC or HPMCAS.

In some embodiments, the surfactant is sodium laurel sulfate or Vitamin E TPGS.

In some embodiments, the VX-950 makes up about 55% to about 70% wt/wt of the pharmaceutical composition, the surfactant is sodium laurel sulfate or Vitamin E TPGS and makes up about 1% wt/wt of the pharmaceutical composition, and the plurality of polymers comprises HPMC and HPMCAS, makes up about 44% to about 29% wt/wt of the pharmaceutical composition, thereby totaling 100% wt/wt of the composition.

In some embodiments, the VX-950 makes up about 55% wt/wt of the pharmaceutical composition, the plurality of polymers makes up about 44% wt/wt of the pharmaceutical composition, and the surfactant is sodium laurel sulfate or Vitamin E TPGS and makes up about 1% wt/wt of the pharmaceutical composition. In some embodiments, the plurality of polymers includes about 55.5% wt/wt HPMCAS and about 44.5% wt/wt HPMC.

In some embodiments, the VX-950 makes up about 55% wt/wt of the pharmaceutical composition, the plurality of polymers makes up about 44% wt/wt of the pharmaceutical composition, and the surfactant is sodium laurel sulfate or Vitamin E TPGS and makes up about 1% wt/wt of the pharmaceutical composition. In some embodiments, the plurality of polymers includes about 33% wt/wt HPMCAS and about 67% wt/wt HPMC.

In some embodiments, the VX-950 makes up about 60% wt/wt of the pharmaceutical composition, the plurality of polymers makes up about 39% wt/wt of the pharmaceutical composition, and the surfactant is sodium laurel sulfate or Vitamin E TPGS and makes up about 1% wt/wt of the pharmaceutical composition. In some embodiments, the plurality of polymers includes about 63% wt/wt HPMCAS and about 36% wt/wt HPMC.

In some embodiments, the VX-950 makes up about 65% wt/wt of the pharmaceutical composition, the plurality of polymers makes up about 34% wt/wt of the pharmaceutical composition, and the surfactant is sodium laurel sulfate or Vitamin E TPGS and makes up about 1% wt/wt of the pharmaceutical composition. In some embodiments, the plurality of polymers includes about 50% wt/wt HPMCAS and about 50% wt/wt HPMC.

In some embodiments, the VX-950 makes up about 70% wt/wt of the pharmaceutical composition, the plurality of polymers makes up about 29% wt/wt of the pharmaceutical composition, and the surfactant is sodium laurel sulfate or Vitamin E TPGS and makes up about 1% wt/wt of the pharmaceutical composition. In some embodiments, the plurality of polymers includes about 33% wt/wt HPMCAS and about 67% wt/wt HPMC.

In another aspect, the disclosure features a pharmaceutical composition including an amorphous VX-950 as a solid dispersion and one or more of a surfactant, polymer, inert pharmaceutically acceptable substance, or pharmaceutically acceptable carrier, e.g., as described herein.

In a preferred embodiment, the solid dispersion exhibits a predetermined level of physical and/or chemical stability. E.g., the solid dispersion retains about 50%, about 60%, about 70%, about 80%, about 90%, about 95%, about 98%, or about 99%, of amorphous VX-950 when stored at 25° C. in a closed water tight container, e.g., an amber glass vial or high density polyethylene (HDPE) container.

In a preferred embodiment, the solid dispersion exhibits at least about 50%, at least about 60%, at least about 70%, at least about 80%, at least about 90%, at least about 95%, at least about 98%, or at least about 99% of the bioavailability when administered to a fed subject as when administered to a fasted subject.

In a preferred embodiment, the solid dispersion exhibits at least about 50%, at least about 60%, at least about 70%, at least about 80%, at least about 90%, at least about 95%, at least about 98%, or at least about 99% of the bioavailability when administered to a fasted subject as when administered to a fed subject.

In some embodiments, the composition includes a mixture of polymers and the polymer mixture includes one or more than one water-soluble polymer or partially water-soluble polymer.

In some embodiments, the VX-950 has improved physical or chemical stability relative to crystalline VX-950. In some embodiments, the solid dispersion has a higher glass transition temperature than the glass transition temperature of neat amorphous VX-950. In some embodiments, the VX-950 has a relaxation rate that is lower than the relaxation rate of neat amorphous VX-950.

In some embodiments, the mixture of polymers includes a cellulosic polymer such as HPMC or HPMCAS.

In some embodiments, the mixture of polymers includes HPMC and/or HPMCAS.

In some embodiments, the pharmaceutical composition also includes a surfactant, either in the solution or as a component of the VX-950 particles or both. The surfactant can be, for example, SLS or vitamin E or a derivative thereof (e.g., vitamin E TPGS).

Methods of preparing a form, dispersion, composition, or formulation described herein.

Accordingly, a process for preparing an amorphous form of VX-950 including spray-drying is described. One embodiment provides a process preparing an amorphous form of VX-950 by combining VX-950 and a suitable solvent to form a mixture and then spray-drying the mixture to obtain the amorphous form of VX-950. The mixture may be either a solution or a suspension.

In another aspect, the disclosure features a solid dispersion prepared according to a process described herein.

This disclosure also provides a process for preparing a solid dispersion of VX-950 comprising:

a) forming a solution of VX-950, a mixture of polymers (e.g., crystallization inhibiting or a stabilizing polymer), and a solvent;

b) rapidly removing the solvent from the solution to form a solid amorphous dispersion comprising VX-950 and the crystallization inhibiting mixture of polymers. In certain embodiments, the solvent is removed by spray drying.

As would be appreciated spray drying may be done and is often done in the presence of an inert gas. In certain embodiments, processes that involve spray drying may be done in the presence of a supercritical fluid involving carbon dioxide or a mixture of carbon dioxide.

Accordingly, in another embodiment, this disclosure provides a process for preparing a solid dispersion of VX-950 comprising

a) forming a mixture of VX-950, mixture of polymers (e.g., one or more of the following: a supporting polymer, a crystallization inhibiting polymer, or stabilizing polymer), and a solvent(or mixture of solvents); and

b) spray-drying the mixture to form a solid dispersion comprising VX-950.

Post-drying and/or polishing the wet spray dried dispersion to below ICH or given specifications for residual solvents can optionally be performed.

These processes could be used to prepare the compositions of this disclosure. The amounts and the features of the components used in the processes would be as described herein.

In some embodiments, the process for preparing a solid dispersion containing an amorphous form of VX-950 and a plurality of polymers includes: spray-drying VX-950 and the plurality of polymers to provide the solid dispersion of VX-950.

In some embodiments, the process includes combining the VX-950, the plurality of polymers, and a suitable solvent to form a mixture and then spray-drying the mixture to obtain the solid dispersion of VX-950.

In some embodiments, the process includes a) forming a mixture comprising VX-950, the plurality of polymers, and a solvent; and

b) spray-drying the mixture to form a solid dispersion comprising VX-950.

In some embodiments, the plurality of polymers comprises HPMC and/or HPMCAS.

In some embodiments, the plurality of polymers is present in an amount of from about 20% to about 60% by weight in the solid dispersion.

In some embodiments, the mixture of polymers is present in an amount of from about 30% to about 70% by weight in the solid dispersion.

In some embodiments, the mixture also includes a surfactant, for example, sodium lauryl sulfate (SLS) or vitamin E or a derivative thereof (e.g., vitamin E TPGS).

In some embodiments, the solvent includes methylene chloride. In some embodiments, the solvent includes acetone. In some embodiments, the solvent includes a mixture of methylene chloride and acetone. For example, the solvent can include from about 0% to about 30% acetone and from about 70% to about 100% methylene chloride, or the solvent can includes from about 0% to about 40% acetone and from about 60% to about 100% methylene chloride. Other exemplary ratios of methylene chloride to acetone include 80:20, 75:25, 70:30, and 60:40.

In some embodiments, the mixture further includes a surfactant, e.g., sodium lauryl sulfate (SLS) or Vitamin E TPGS.

In some embodiments, the solvent includes methylene chloride.

In some embodiments, the solvent includes acetone.

In some embodiments, the solvent includes from about 0% to about 30% acetone and from about 70% to about 100% methylene chloride.

In some embodiments, the solvent includes from about 0% to about 40% acetone and from about 60% to about 100% methylene chloride.

In some embodiments, a solid dispersion prepared according to a process described herein.

In one aspect, the disclosure features a method for treating HCV infection in a mammal comprising administering a solid dispersion described herein

In one aspect, the disclosure features a pharmaceutical pack or kit that includes the solid dispersion of VX-950 disclosed herein.

In one aspect, the disclosure features an oral formulation (e.g., tablet) that includes the solid dispersion of VX-950 disclosed herein.

In another aspect, the disclosure features a method of treating HCV infection in a mammal. In one embodiment, the method includes administering amorphous VX-950, wherein the amorphous VX-950 is as defined herein. In another embodiment, the method includes administering a solid dispersion described herein.

In another embodiment, the method includes administering an additional agent selected from an immunomodulatory agent; an antiviral agent; another inhibitor of HCV NS3/4A protease; another inhibitor of IMPDH; an inhibitor of a target in the HCV life cycle other than NS3/4A protease; an inhibitor of internal ribosome entry, a broad-spectrum viral inhibitor; a cytochrome P-450 inhibitor; or combinations thereof.

In another aspect, the disclosure features pharmaceutical packs or kits including a VX-950 composition described herein or amorphous VX-950.

An amorphous form of a drug may exhibit different properties than the crystalline form (see, U.S. Pat. No. 6,627,760). Embodiments of the disclosure include amorphous VX-950, which thermodynamically is at a higher energy level than its corresponding crystalline form. Therefore, it is energetically more active, and thus often exhibits higher metastable solubility, faster dissolution behavior, as well as less stable physical properties. The first two properties act to enhance the aqueous solubility and bioavailability of the drug, while the last may be detrimental to this goal by presenting a physically less stable composition, of which the bioavailability may change due to recrystallization of the drug from its amorphous state during storage, or upon administration to humans or animals.

To improve the stability of an amorphous solid (which is generally less stable than a crystal form), a mixture of polymers can be used to form an amorphous solid dispersion system together with the drug.

The manufacture of an amorphous solid dispersion containing VX-950 presented several challenges. First, VX-950 does not dissolve to a significant amount in water or most other conventional organic solvents, including acetone, ethyl acetate, and acetonitrile. The aqueous solubility of VX-950 at room temperature is virtually undetectable by HPLC and the aqueous solubility is not pH-dependent. Second, VX-950 has shown chemical reactivity with some alcohols, for example, MeOH, EtOH, and iPrOH, which makes these unsuitable solvents. Third, the melting point of VX-950 is about 240° C., making hot-melt technologies somewhat impractical due to the potential degradation of VX-950 at the high temperature. Therefore, an appropriate solvent or solvent mixture is crucial to optimizing the processing and production of a solid dispersion.

Amorphous solid dispersions of the disclosure can significantly improve the oral bioavailability of VX-950. In the presence of an appropriate surfactant or surfactant mixture (e.g., SLS or Vitamin E d-alpha tocopheryl polyethylene glycol 1000 succinate (Vitamin E TPGS)), the bioavailability can be further enhanced.

Amorphous solid dispersions of the disclosure can provide improved bioavailability of VX-950 when orally administered relative to the administration of crystalline VX-950. In some embodiments, these solid dispersions are in a solid state that can be conveniently stored and administered. The manufacture of the solid dispersions can be conducted and scaled up successfully by selecting an organic solvent or solvent mixture (for example, methylene chloride, acetone, etc.) or a supercritical fluid (for example, involving carbon dioxide). In some embodiments, solid dispersions can have improved chemical and physical stability. For example, in some instances the solid dispersions can be chemically and/or physically stable for at least two years at conventional storage conditions (room temperature).

The inventors have discovered that varying the solvent, for example including a non-volatile or high boiling solvent, during spray drying of a drug or other therapeutic agent (e.g., a solid dispersion of the drug or therapeutic agent) can improve the properties of the resulting product (e.g., a solid dispersion such as an amorphous solid dispersion of the drug or therapeutic agent). In some instances, including a non-volatile or high boiling solvent as a component of a solvent mixture in the spray drying process can result in an increase in the amount of time required for the resulting particles to solidify and/or dry, thereby in some instances providing improved particles, e.g., particles that are larger and/or denser and/or more flowable than the same particles had they been obtained using a solvent system without a non-volatile or high boiling solvent.

In one aspect, the method includes a method of spray drying a drug or other therapeutic agent, the method comprising forming a mixture of the drug in a suitable solvent or combination of solvents where at least one solvent is a non-volatile or high boiling solvent to form a mixture of the drug and solvent, and then spray-drying the mixture to obtain amorphous drug product. The mixture can be either a solution or a suspension.

In some embodiments, the drug is a small molecule drug, for example a drug having a molecular weight of less than about 1000 daltons, e.g., less than about 750 daltons or less than about 500 daltons.

In some embodiments, the drug is a poorly soluble drug.

The drug can be selected from one of the following classifications: analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opiod analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, or non-essential fatty acids.

In some preferred embodiments, the drug is an anti-viral agent, for example an antiviral agent used to treat Hepatitis C (HepC), such as a HepC protease inhibitor. In some most preferred embodiments, the drug is VX-950:

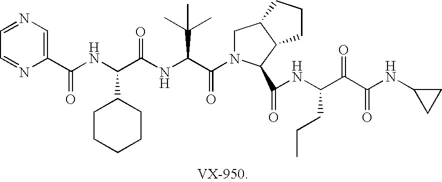

In some embodiments, the solvent is a combination of solvent components including at least one non-volatile solvent. For example, the solvent is a combination of components that includes both a volatile solvent and a non-volatile solvent.

Examples of suitable volatile solvents include those that dissolve or suspend the drug either alone or in combination with another co-solvent. In some preferred examples, the solvent or solvent combination completely dissolves the drug.

Examples of volatile solvents include methylene chloride, acetone, chloroform, and THF. Examples of non-volatile solvents include organic acids such as glacial acetic acid, DMSO, DMF, or water.

In some embodiments, the non-volatile solvent is a component in a solvent system. For example the non-volatile solvent is present as a component in a solvent from about 1% to about 20% by wt (e.g., from about 3% to about 15%, from about 4% to about 12%, or from about 5% to about 10%).

In some preferred embodiments, the solvent system is a combination of a volatile solvent or combination of solvents such as methylene chloride and acetone with a non volatile solvent such as glacial acetic acid. For example, the solvent system comprises from about 40% to about 80% methylene chloride, from about 20% to about 35% acetone, and from about 1% to about 15% glacial acetic acid (e.g., from about 50% to about 70% methylene chloride, from about 25% to about 30% acetone, and from about 3% to about 12% glacial acetic acid).

In some embodiments, the solvent system comprises glacial acetic acid.

In some embodiments, the solvent systems comprises a combination of glacial acetic acid with at least one volatile solvent such as acetone and/or methylene chloride (e.g., a mixture of methylene chloride and acetone).

In some embodiments, the mixture also includes a surfactant, for example, sodium lauryl sulfate (SLS) or Vitamin E or a derivative thereof (e.g., Vitamin E TPGS).

In some preferred embodiments, the solvent system is a combination of a volatile solvent or combination of solvents such as methylene chloride and acetone with a non-volatile solvent such as water. For example, the solvent system comprises from about 40% to about 80% methylene chloride, from about 20% to about 35% acetone, and from about 0.1% to about 15% water (e.g., from about 50% to about 70% methylene chloride, from about 25% to about 30% acetone, and from about 1% to about 5% water).

In some embodiments, the solvent system comprises water.

In some embodiments, the solvent system comprises a combination of water with at least one volatile solvent such as acetone and/or methylene chloride (e.g., a mixture of methylene chloride and acetone).

In some embodiments, the mixture also includes a surfactant, for example, sodium lauryl sulfate (SLS) or Vitamin E or a derivative thereof (e.g., Vitamin E TPGS).

In another aspect, the method of spray drying includes forming a solid dispersion of a drug and one or more polymers comprising forming or providing a mixture of the drug and the polymer(s) in a suitable solvent or combination of solvents where at least one solvent is a non-volatile or high boiling solvent to form a mixture of the drug, polymer(s) and solvent, and then spray-drying the mixture to obtain a solid dispersion drug product. The mixture can be either a solution or a suspension. In a preferred embodiment, the solid dispersion product is an amorphous solid dispersion. For example, an amorphous solid dispersion that is substantially free of crystalline drug product.

Examples of polymers for the solid dispersion include one or more water-soluble polymer(s) or partially water-soluble polymer(s). Water-soluble or partially water-soluble polymers include but are not limited to, cellulose derivatives (e.g., hydroxypropylmethylcellulose (HPMC), hydroxypropylcellulose (HPC)) or ethylcellulose; polyvinylpyrrolidones (PVP); polyethylene glycols (PEG); polyvinyl alcohols (PVA); acrylates, such as polymethacrylate (e.g., Eudragit® E); cyclodextrins (e.g., β-cyclodextin) and copolymers and derivatives thereof, including for example PVP-VA (polyvinylpyrollidone-vinyl acetate).

In some preferred embodiments, the polymer is hydroxypropylmethylcellulose (HPMC), such as HMPC60SH50, HPMC E50 or HPMCE15.

In some embodiments, the polymer is a pH-dependent enteric polymer. Such pH-dependent enteric polymers include, but are not limited to, cellulose derivatives (e.g., cellulose acetate phthalate (CAP)), hydroxypropyl methyl cellulose phthalates (HPMCP), hydroxypropyl methyl cellulose acetate succinate (HPMCAS), carboxymethylcellulose (CMC) or a salt thereof (e.g., a sodium salt such as (CMC-Na)); cellulose acetate trimellitate (CAT), hydroxypropylcellulose acetate phthalate (HPCAP), hydroxypropylmethyl-cellulose acetate phthalate (HPMCAP), and methylcellulose acetate phthalate (MCAP), or polymethacrylates (e.g., Eudragit® S).

In some preferred embodiments, the polymer is hydroxypropyl methyl cellulose acetate succinate (HPMCAS), e.g., HMPC AS-HG.

In another embodiment, the polymer(s) is an insoluble cross-linked polymer, for example a polyvinylpyrrolidone (e.g., Crospovidone).

In another embodiment, the polymer(s) is polyvinylpyrrolidone (PVP).

In some embodiments, the polymer is a mixture of two or more polymers (e.g., a combination of two cellulosic polymers such as HPMC and HPMCAS).

In some embodiments, the polymer(s) is present in an amount of from about 30% to about 70% by weight in the solid dispersion.

In some embodiments the drug is a small molecule drug, for example a drug having a molecular weight of less than about 1000 daltons, e.g., less than about 750 daltons or less than about 500 daltons.

In some embodiments, the drug is a poorly soluble drug.

The drug can be selected from one of the following classifications: analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opiod analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, or non-essential fatty acids.

In some preferred embodiments, the drug is an anti-viral agent, for example an antiviral agent used to treat HepC, such as a HepC protease inhibitor. In some most preferred embodiments, the drug is VX-950:

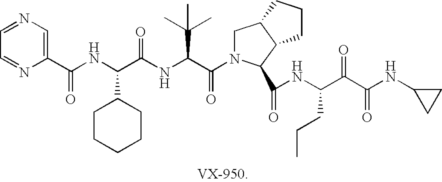

In some embodiments, the solvent is a combination of solvent components including at least one non-volatile solvent. For example, the solvent is a combination of components that includes both a volatile solvent and a non-volatile solvent.

Examples of suitable volatile solvents include those that dissolve or suspend the drug either alone or in combination with another co-solvent. In some preferred examples, the solvent or solvent combination completely dissolves the drug.

Examples of volatile solvents include methylene chloride, acetone, chloroform, and THF. Examples of non-volatile solvents include organic acids such as glacial acetic acid, DMSO, DMF, or water.

In some embodiments, the non-volatile solvent is a component in a solvent system. For example the non-volatile solvent is present as a component in a solvent from about 1% to about 20% by wt (e.g., from about 3% to about 15%, from about 4% to about 12%, or from about 5% to about 10%).

In some preferred embodiments, the solvent system is a combination of a volatile solvent or combination of solvents such as methylene chloride and acetone with a non volatile solvent such as glacial acetic acid. For example, the solvent system comprises from about 40% to about 80% methylene chloride, from about 20% to about 35% acetone, and from about 1% to about 15% glacial acetic acid (e.g., from about 50% to about 70% methylene chloride, from about 25% to about 30% acetone, and from about 3% to about 12% glacial acetic acid).

In some preferred embodiments, the solvent system is a combination of a volatile solvent or combination of solvents such as methylene chloride and acetone with a non-volatile solvent such as water. For example, the solvent system comprises from about 40% to about 80% methylene chloride, from about 20% to about 35% acetone, and from about 0.1% to about 15% water (e.g., from about 50% to about 70% methylene chloride, from about 25% to about 30% acetone, and from about 1% to about 5% water).

In some embodiments, the mixture also includes a surfactant, for example, sodium lauryl sulfate (SLS) or Vitamin E or a derivative thereof (e.g., Vitamin E TPGS).

In another aspect, the process includes

a) forming or providing a mixture of a poorly water soluble drug, at least one polymer, and a solvent system comprising at least one non-volatile solvent; and

b) spray-drying the mixture to form a solid dispersion comprising a poorly water soluble drug to obtain a solid dispersion of the drug.

In some embodiments the drug is a small molecule drug, for example a drug having a molecular weight of less than about 1000 daltons, e.g., less than about 750 daltons or less than about 500 daltons.

The drug can be selected from one of the following classifications: analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opiod analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, or non-essential fatty acids.

In some preferred embodiments, the drug is an anti-viral agent, for example an antiviral agent used to treat HepC, such as a HepC protease inhibitor. In some most preferred embodiments, the drug is VX-950:

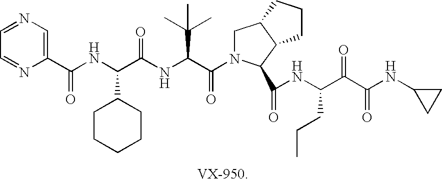

In some embodiments, the solvent is a combination of solvents including at least one non-volatile solvent. For example, the solvent is a combination of components that includes both a volatile solvent and a non-volatile solvent.

Examples of suitable volatile solvents include those that dissolve or suspend the drug either alone or in combination with another co-solvent. In some preferred examples, the solvent or solvent combination completely dissolves the drug.

Examples of volatile solvents include methylene chloride, acetone, chloroform, THF.

Examples of non-volatile solvents include organic acids such as glacial acetic acid, DMSO, DMF, or water.

In some embodiments, the non-volatile solvent is a component in a solvent system. For example the non-volatile solvent is present as a component in a solvent from about 1% to about 20% by wt (e.g., from about 3% to about 15%, from about 4% to about 12%, or from about 5% to about 10%).

In some preferred embodiments, the solvent system is a combination of a volatile solvent or combination of solvents such as methylene chloride and acetone with a non volatile solvent such as glacial acetic acid. For example, the solvent system comprises from about 40% to about 80% methylene chloride, from about 20% to about 35% acetone, and from about 1% to about 15% glacial acetic acid (e.g., from about 50% to about 70% methylene chloride, from about 25% to about 30% acetone, and from about 3% to about 12% glacial acetic acid).

In some preferred embodiments, the solvent system is a combination of a volatile solvent or combination of solvents such as methylene chloride and acetone with a non-volatile solvent such as water. For example, the solvent system comprises from about 40% to about 80% methylene chloride, from about 20% to about 35% acetone, and from about 0.1% to about 15% water (e.g., from about 50% to about 70% methylene chloride, from about 25% to about 30% acetone, and from about 1% to about 5% water).

In some embodiments, the mixture also includes a surfactant, for example, sodium lauryl sulfate (SLS) or Vitamin E or a derivative thereof (e.g., Vitamin E TPGS).

Examples of polymers for the solid dispersion include one or more water-soluble polymer(s) or partially water-soluble polymer(s). Water-soluble or partially water-soluble polymers include but are not limited to, cellulose derivatives (e.g., hydroxypropylmethylcellulose (HPMC), hydroxypropylcellulose (HPC)) or ethylcellulose; polyvinylpyrrolidones (PVP); polyethylene glycols (PEG); polyvinyl alcohols (PVA); acrylates, such as polymethacrylate (e.g., Eudragit® E); cyclodextrins (e.g., β-cyclodextin) and copolymers and derivatives thereof, including for example PVP-VA (polyvinylpyrollidone-vinyl acetate).

In some preferred embodiments, the polymer is hydroxypropylmethylcellulose (HPMC), such as HPMC60SH50, HPMC E50 or HPMCE15.

In some embodiments, the polymer is a pH-dependent enteric polymer. Such pH-dependent enteric polymers include, but are not limited to, cellulose derivatives (e.g., cellulose acetate phthalate (CAP)), hydroxypropyl methyl cellulose phthalates (HPMCP), hydroxypropyl methyl cellulose acetate succinate (HPMCAS), carboxymethylcellulose (CMC) or a salt thereof (e.g., a sodium salt such as (CMC-Na)); cellulose acetate trimellitate (CAT), hydroxypropylcellulose acetate phthalate (HPCAP), hydroxypropylmethyl-cellulose acetate phthalate (HPMCAP), and methylcellulose acetate phthalate (MCAP), or polymethacrylates (e.g., Eudragit® S).

In some preferred embodiments, the polymer is hydroxypropyl methyl cellulose acetate succinate (HPMCAS), e.g., HPMC AS-HG.

In another embodiment, the polymer(s) is an insoluble cross-linked polymer, for example a polyvinylpyrrolidone (e.g., Crospovidone).

In another embodiment, the polymer(s) is polyvinylpyrrolidone (PVP).

In some embodiments, the polymer is a mixture of two or more polymers (e.g., a combination of two cellulosic polymers such as HPMC and HPMCAS).

In some embodiments, the polymer(s) is present in an amount of from about 30% to about 70% by weight in the solid dispersion.

In some embodiments, the mixture also includes a surfactant, for example, sodium lauryl sulfate (SLS) or Vitamin E or a derivative thereof (e.g., Vitamin E TPGS).

In another aspect, this disclosure provides a process for preparing a solid dispersion of VX-950 comprising:

a) forming or provising a solution of VX-950, a cellulosic polymer, and a solvent, wherein the solvent comprises at least one non-volatile solvent component (e.g., glacial acetic acid);

b) spray-drying the mixture to form a solid amorphous dispersion comprising VX-950 and the cellulosic polymer.

In some embodiments, the polymer is HPMC, HPMCAS, or a mixture thereof. In some preferred embodiments, the polymer is HPMCAS or a mixture of HPMC and HPMCAS.

Examples of suitable volatile solvents include those that dissolve or suspend the drug either alone or in combination with another co-solvent. In some preferred examples, the solvent or solvent combination completely dissolves the drug.

Examples of volatile solvents include methylene chloride, acetone, chloroform, THF.

Examples of non-volatile solvents include organic acids such as glacial acetic acid, DMSO, DMF, or water.

In some embodiments, the non-volatile solvent is a component in a solvent system. For example the non-volatile solvent is present as a component in a solvent from about 1% to about 20% by wt (e.g., from about 3% to about 15%, from about 4% to about 12%, or from about 5% to about 10%).

In some preferred embodiments, the solvent system is a combination of a volatile solvent or combination of solvents such as methylene chloride and acetone with a non volatile solvent such as glacial acetic acid. For example, the solvent system comprises from about 40% to about 80% methylene chloride, from about 20% to about 35% acetone, and from about 1% to about 15% glacial acetic acid (e.g., from about 50% to about 70% methylene chloride, from about 25% to about 30% acetone, and from about 3% to about 12% glacial acetic acid).

In some preferred embodiments, the solvent system is a combination of a volatile solvent or combination of solvents such as methylene chloride and acetone with a non-volatile solvent such as water. For example, the solvent system comprises from about 40% to about 80% methylene chloride, from about 20% to about 35% acetone, and from about 0.1% to about 15% water (e.g., from about 50% to about 70% methylene chloride, from about 25% to about 30% acetone, and from about 1% to about 5% water).

In some embodiments, the mixture also includes a surfactant, for example, sodium lauryl sulfate (SLS) or Vitamin E or a derivative thereof (e.g., Vitamin E TPGS).

In some embodiments, the solvent comprises a mixture of methylene chloride, acetone, and glacial acetic acid.

In another aspect, this disclosure provides a process for preparing a solid dispersion of VX-950 comprising

a) forming or providing a mixture of VX-950, at least one cellulosic polymer, and a solvent wherein the solvent comprises glacial acetic acid; and

b) spray-drying the mixture to form a solid dispersion comprising VX-950.

In some embodiments, the polymer is HPMC, HPMCAS, or a mixture thereof In some preferred embodiments, the polymer is HPMCAS or a mixture of HPMC and HPMCAS.

In some embodiments, the solvent also comprises a volatile solvent or combination of solvents that dissolve or suspend the drug and polymer. In some preferred examples, the solvent or solvent combination completely dissolves the drug and polymer.

In some preferred embodiments, the solvent includes a mixture of methylene chloride and acetone.

In some embodiments, the glacial acetic acid is present as a component in a solvent from about 1% to about 20% by wt (e.g., from about 3% to about 15%, from about 4% to about 12%, or from about 5% to about 10%).

In some embodiments, the solvent comprises a mixture of methylene chloride, acetone, and glacial acetic acid.

In some embodiments, the solvent system comprises from about 40% to about 80% methylene chloride, from about 20% to about 35% acetone, and from about 1% to about 15% glacial acetic acid (e.g., from about 50% to about 70% methylene chloride, from about 25° A to about 30% acetone, and from about 3% to about 12% glacial acetic acid).

In some preferred embodiments, the solvent system comprises from about 40% to about 80% methylene chloride, from about 20% to about 35% acetone, and from about 0.1% to about 15% water (e.g., from about 50% to about 70% methylene chloride, from about 25% to about 30% acetone, and from about 1% to about 5% water).

In some embodiments, the mixture also includes a surfactant, for example, sodium lauryl sulfate (SLS) or Vitamin E or a derivative thereof (e.g., Vitamin E TPGS).

In one aspect, the disclosure provides product made by a process described herein. For example a solid dispersion of a drug (e.g., VX-950), such as an amorphous solid dispersion of a drug (e.g., VX-590). For example an amorphous solid dispersion including a drug (e.g., VX-950), at least one polymer, and optionally one or more solubility enhancing surfactant (e.g., SLS or Vitamin E TPGS) is provided. The dispersion can enhance the aqueous solubility and bioavailability of the drug (e.g., VX-950) upon oral dosing of the solid dispersion to a mammal (e.g., a rat, dog or human). In certain aspects, at least a portion of the drug (e.g., VX-950) in the solid dispersion is in the amorphous state (e.g., at least about 50%, at least about 55%, at least about 60%, at least about 65%, at least about 70%, at least about 75%, at least about 80%, at least about 85%, at least about 90%, at least about 95%, at least about 98%, or at least about 99%). In preferred embodiments, the solid dispersion is essentially or substantially free of crystalline drug (e.g., VX-950).

As would be appreciated, spray drying may be done in the presence of an inert gas. In certain embodiments, processes that involve spray drying may be done in the presence of a supercritical fluid involving carbon dioxide or a mixture of carbon dioxide.

A “poorly soluble drug” as used herein means drugs that are essentially totally water-insoluble or sparingly water-soluble. The term applies to any beneficial therapeutic agent having a dose (mg) to aqueous solubility (mg/ml) ratio greater than 100 ml, where the drug solubility is that of the neutral (e.g., free base or free acid) form in unbuffered water. This definition includes but is not limited to drugs that have essentially no aqueous solubility (less than 1.0 μg/ml).

All cited patents, patent applications, and references are hereby incorporated by reference in their entireties. In the case of conflict, the present application controls.

The details of one or more embodiments of the disclosure are set forth in the accompanying description below. Other features, objects, and advantages of the disclosure will be apparent from the description and from the claims.

### DESCRIPTION OF DRAWINGS

FIG. 1 depicts a flowchart of a manufacturing process, control, sampling, and testing for the preparation of a spray dried dispersion of which amorphous VX-950 is a component.

FIG. 2 depicts a schematic of a spray drying manufacturing process for the preparation of a spray dried dispersion of which amorphous VX-950 is a component.

FIG3 depicts a line graph showing dissolution rates for various VX-950 solid dispersions in fasted SGF at 37.5° C.

FIG4 depicts a line graph showing dissolution rates for various VX-950 solid dispersions in fasted SGF at 37.5° C.

FIG. 5 depicts a line graph showing dissolution rates for various VX-950 solid dispersions in fasted SGF at 37.5° C.

### DETAILED DESCRIPTION

In general, it has been found that absolute bioavailability after orally administering a micronized crystalline drug powder of VX-950 to rats is less than 0.5%. Simple mixtures of VX-950 with conventional pharmaceutical excipients exhibit similarly low bioavailability upon oral administration to mammals. Compositions including VX-950 in crystalline form (i.e., where a significant portion of VX-950 is in crystalline form) generally do not achieve drug absorption to an extent that provides for sufficient therapeutic effects of VX-950. The compositions described herein provide comparatively improved bioavailability. Accordingly, in some embodiments, a preparation of amorphous VX-950 is provided. For example, a purified preparation that is substantially free of impurities, such as crystalline VX-950, is described. In some embodiments, the disclosure includes a pharmaceutical composition in the form of a solid dispersion comprising VX-950. The compositions of this disclosure are stable, easy to administer, and give high bioavailability of VX-950 upon administration.

In certain embodiments, the VX-950 is present in an amount of from about 5% to about 95% by weight, for example from about 30% to about 90%, preferably up to about 55% (e.g., 53% to 57%), up to about 60% (e.g., 58% to 62%), up to about 65% (e.g., 63% to 67%), up to about 70% (e.g., 68% to 72%), up to about 75% (e.g., 73% to 77%), up to about 80% (e.g., 78% to 82%), up to about 85% (e.g., 83% to 87%), or up to about 90% (e.g., 88% to 92%) by weight. The VX-950 is a mixture of the D-isomer and L-isomer or is a substantially pure product of either isomer. The VX-950 is preferably substantially amorphous (e.g., at least about 50% of VX-950 is amorphous, at least about 55% of VX-950 is amorphous, at least about 60% of VX-950 is amorphous, at least about 65% of VX-950 is amorphous, at least about 70% of VX-950 is amorphous, at least about 75% of VX-950 is amorphous, at least about 80% of VX-950 is amorphous, at least about 85% of VX-950 is amorphous, at least about 90% of VX-950 is amorphous, at least about 95% of VX-950 is amorphous, at least about 98% of VX-950 is amorphous, at least about 99% of VX-950 is amorphous, or substantially all of VX-950 is amorphous.

As used herein, the term “amorphous” refers to a solid material having no long range order in the position of its atoms. Amorphous solids are generally supercooled liquids in which the molecules are arranged in a random manner so that there is no well-defined arrangement and no long range order. Amorphous solids are generally isotropic, i.e., exhibit similar properties in all directions and do not have definite melting points. For example, an amorphous material is a solid material having no sharp characteristic crystalline peak(s) in its X-ray powder diffraction (XRPD) pattern (i.e., is not crystalline as determined by XRPD). Instead, one or several broad peaks (e.g., halos) appear in its XRPD pattern. Broad peaks (e.g., halos) are characteristic of an amorphous solid. See, US 2004/0006237 for a comparison of XRPDs of an amorphous material and crystalline material.

As used herein “crystalline solids” refers to compounds or compositions where the structural units are arranged in fixed geometric patterns or lattices, so that crystalline solids have rigid long range order. The units that constitute the crystal structure can be atoms, molecules, or ions. Crystalline solids show definite melting points.

As used herein, a “dispersion” refers to a disperse system in which one substance, the dispersed phase, is distributed, in discrete units, throughout a second substance (the continuous phase or vehicle). The size of the dispersed phase can vary considerably (e.g., colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include a crystalline drug (dispersed phase) in an amorphous polymer(s) (continuous phase), or alternatively, an amorphous drug (dispersed phase) in an amorphous polymer (continuous phase). In some embodiments, an amorphous solid dispersion includes the polymer(s) (and optionally a surfactant) constituting the dispersed phase, and the drug constitutes the continuous phase.

The term “amorphous solid dispersion” generally refers to a solid dispersion of two or more components, usually a drug and polymer (or plurality of polymers), but possibly containing other components such as surfactants or other pharmaceutical excipients, where the drug is in the amorphous phase, and the physical stability and/or dissolution and/or solubility of the amorphous drug is enhanced by the other components.

A solid dispersion as provided herein is a particularly favorable embodiment of this disclosure. Solid dispersions typically include a compound dispersed in an appropriate carrier medium, such as a solid state carrier. In some embodiments, a carrier according to this disclosure comprises a polymer (e.g., a water-soluble polymer or a partially water-soluble polymer). Preferably, in some embodiments, the carrier comprises a plurality of polymers, preferably, one or more water-soluble polymers or one or more partially water-soluble polymers, or a combination thereof.

An exemplary solid dispersion is a co-precipitate or a co-melt of VX-950 with a plurality of polymers. A “co-precipitate” is a product after dissolving a drug and a plurality of polymers in a solvent or solvent mixture followed by the removal of the solvent or solvent mixture. Sometimes the mixture of polymers can be suspended in the solvent or solvent mixture. The solvent or solvent mixture includes organic solvents and supercritical fluids. The solvent or solvent mixture can also contain a non volatile solvent, such as glacial acetic acid or water. A “co-melt” is a product after heating a drug and a polymer(s) to melt, optionally in the presence of a solvent or solvent mixture, followed by mixing, removal of at least a portion of the solvent if applicable, and cooling to room temperature at a selected rate. In some cases, the solid dispersions are prepared by adding a solution of a drug and solid polymers followed by mixing and removal of the solvent or solvent mixture. To remove the solvent or solvent mixture, vacuum drying, spray drying, tray drying, lyophilization, and other drying procedures may be applied. Applying any of these methods using appropriate processing parameters, according to this disclosure, would provide VX-950 in an amorphous state in the final solid dispersion product.

Production of Amorphous VX-950

Any method for obtaining amorphous forms and solid dispersions could be used in connection with this disclosure including, for example, those described in U.S. Pub. App. No. 2003/0186952 (see the documents cited therein at paragraph 1092) and U.S. Pub. App. No. 2003/0185891). In general, methods that could be used include those that involve rapid removal of solvent or solvent mixture from a mixture or cooling a molten sample. Such methods include, but are not limited to, rotational evaporation, freeze-drying (i.e., lyophilization), vacuum drying, melt congealing, and melt extrusion. However, a preferred embodiment of this disclosure involves amorphous solid dispersion obtained by spray-drying. Accordingly, in another embodiment, this disclosure provides drying the product obtained by spray drying to remove the solvent or solvent mixture.

Preparations disclosed herein, e.g., a pharmaceutical composition, can be obtained by spray-drying a mixture comprising VX-950, a suitable plurality of polymers, and an appropriate solvent or solvent mixture. Spray drying involves atomization of a liquid mixture containing, e.g., a solid and a solvent or solvent mixture, and removal of the solvent or solvent mixture. The solvent or solvent mixture can also contain a non volatile solvent, such as glacial acetic acid. Atomization may be done, for example, through a two-fluid or pressure or electrosonic nozzle or on a rotating disk.

Spray drying converts a liquid feed to a dried particulate form. Optionally, a secondary drying process such as fluidized bed drying or vacuum drying, may be used to reduce residual solvents (and other additives, such as glacial acetic acid) to pharmaceutically acceptable levels. Typically, spray-drying involves contacting a highly dispersed liquid suspension or solution (e.g., atomized solution), and a sufficient volume of hot air or gas (e.g., nitrogen, e.g., pure nitrogen) to produce evaporation and drying of the liquid droplets. The preparation to be spray dried can be any solution, coarse suspension, slurry, colloidal dispersion, or paste that may be atomized using the selected spray-drying apparatus. In a standard procedure, the preparation is sprayed into a current of warm filtered air (or into gas, e.g., nitrogen) that evaporates the solvent and conveys the dried product to a collector (e.g., a cyclone). The spent air or gas is then exhausted with the solvent (or solvent mixture including any additives such as glacial acetic acid), (e.g., then filtered) or alternatively the spent air or gas is sent to a condenser to capture and potentially recycle the solvent or solvent mixture. For example, if a gas (e.g., nitrogen) is used, the gas is then optionally recycled, heated again and returned to the unit in a closed loop system. Commercially available types of apparatus may be used to conduct the spray-drying. For example, commercial spray dryers are manufactured by Buchi Ltd. and Niro (e.g., the PSD line of spray driers manufactured by Niro) (see, U.S. Pub. App. Nos. 2004/0105820 and 2003/0144257).

Spray-drying typically employs solids loads of material from about 1% to about 30% or up to about 50% (i.e., drug plus and excipients), preferably at least about 10%. In some embodiments, solids loads of less than 10% may result in poor yields and unacceptably long run-times. In general, the upper limit of solids loads is governed by the viscosity of (e.g., the ability to pump) the resulting solution and the solubility of the components in the solution. Generally, the viscosity of the solution can determine the size of the particle in the resulting powder product.

Techniques and methods for spray-drying may be found in Perry's Chemical Engineering Handbook, 6th Ed., R. H. Perry, D. W. Green & J. O. Maloney, eds., McGraw-Hill Book Co. (1984); and Marshall “Atomization and Spray-Drying” 50, Chem. Eng. Prog. Monogr. Series 2 (1954). In general, the spray-drying is conducted with an inlet temperature of from about 40° C. to about 200° C., for example, from about 70° C. to about 150° C., preferably from about 40° C. to about 60° C., about 50° C. to about 55° C., 80° C. to about 110° C., e.g., about 90° C. The spray-drying is generally conducted with an outlet temperature of from about 20° C. to about 100° C., for example from about 25° C. to about 30° C. (e.g., about 26° C.), about 40° C. to about 50° C., about 50° C. to about e.g., about 56° C. or 58° C.

Removal of the solvent or solvent mixture may require a subsequent drying step, such as tray drying, fluid bed drying (e.g., from about room temperature to about 100° C.), vacuum drying, microwave drying, rotary drum drying or biconical vacuum drying (e.g., from about room temperature to about 200° C.).

The inventors have found that there is a direct relationship between bulk density/flow and residual solvent(s); the higher the bulk density/better flow, the higher the residual solvent(s). It may be advantageous to optimize the powder flow and bulk density and use secondary drying to remove the residual solvent or solvent mixture. In one embodiment of this disclosure, the solid dispersion is fluid-bed dried. Fluid-bed drying at about 75° C. for about 8 hours has been found effective in certain embodiments to provide optimal effects in certain solid dispersions of VX-950. In other embodiments, e.g. using HPMCAS in the plurality of polymers in the solid dispersion, fluid-bed drying at 45° C. for about 4 hours has been effective to provide acceptable levels of residual solvent in the final product.

In preferred processes, the solvent includes a volatile solvent. In some embodiments, the solvent includes a mixture of volatile solvents. Preferable solvents include those that can dissolve both VX-950 and the polymers. Suitable solvents include those described above, for example, methylene chloride, acetone, etc. In more preferred processes, the solvent is a mixture of methylene chloride and acetone. The percent weight ratio of methylene chloride:acetone can be for example, about 100:0, about 90:10, about 80:20, about 70:30, about 60:40, and is preferably about 80:20 or about 70:30. The solvent or solvent mixture can also contain a non volatile solvent, such as glacial acetic acid. The organic acid or polar solvent solvent can be, e.g., up to about 5%, up to about 10%, or up to about 15% by weight of the solvent mixture. For example, a solvent mixture can contain a percent weight ratio of methylene chloride:acetone:glacial acetic acid of about 67:28:5 or 63:27:10. Although alcoholic solvents could be used in connection with this disclosure, alcohols have been found to react with VX-950 to form ketals. Accordingly, a solvent that does not react with VX-950 (particularly to form ketals) is preferred. Such a solvent should not contain an OH group or a similarly reactive moiety. In these processes, therefore, a preferred solvent is other than an alcohol.

Because of the reactivity of VX-950, a preferred polymer for use in a plurality of polymers in connection with this disclosure is other than a polyethylene glycol (e.g., PEG 8000) (i.e., other than a polymer having free hydroxyl moieties).

The particle size and the temperature drying range may be modified to prepare an optimal solid dispersion. As would be appreciated by skilled practitioners, a small particle size would lead to improved solvent removal. Applicants have found, however, that smaller particles lead to fluffy particles that do not provide optimal solid dispersions of VX-950 for downstream processing such as tableting. At higher temperatures, crystallization or chemical degradation of VX-950 may occur. At lower temperatures, a sufficient amount of the solvent(s) may not be removed. The methods herein provide a optimal particle size and an optimal drying temperature. Further, the applicants have found that the addition of a non volatile solvent, such as glacial acetic acid, to the solvent or solvent mixture can result in larger, denser, and more flowable particles. Such particles may be better suited for downstream processes, such as compression into tablets.

Polymers

Solid dispersions including VX-950 and a plurality of polymers (or solid state carrier(s)) are provided herein. A polymeric mixture of a plurality of polymers can be used as part of an amorphous solid dispersion system together with the drug. Without being bound by theory, the presence of a plurality of polymers can help prevent, decrease, or slow the amount or rate of crystallization of the drug as compared to the amount or rate of crystallization that occurs in the absence of a polymer. For example, the amount of crystallization when a plurality of polymers is used can be decreased by at least about 10%, by at least about 20%, by at least about 30%, by at least about 40%, by at least about 50%, by at least about 60%, by at least about 70%, by at least about 80%, by at least about 90%, by at least about 95%, or by at least about 99% compared to the amount of crystallization in the absence of a polymer. For example, a plurality of polymers can protect a drug against crystallization in an aqueous medium, such as gastric fluids and/or in intestinal fluids. For example, HPMC can help decrease the amount of crystallization (e.g., of VX-950) in low pH, such as in gastric fluids. HPMC can provide protection in gastric fluids (e.g., fasted or fed gastric fluids), and simulated gastric fluids (“SGF”) (e.g., fasted or fed SGF). As another example, HPMCAS can provide increased physical stability and decrease the amount of crystallization (e.g., of VX-950) in intestinal fluids (e.g., fasted or fed intestinal fluids) and simulated intestinal fluids (“SIF”) (e.g., fasted or fed SIF). As a result, one or more of bioavailability, solubility and absorption of VX-950 can be enhanced. In addition, by decreasing the rate of crystallization, a plurality of polymers can increase the shelf stability of a composition, e.g., a spray dried dispersion or a solid form (e.g., a tablet), containing VX-950 relative to the stability of the composition when no polymer is used by at least about 10% (e.g., by at least about 20%, by at least about 30%, by at least about 40%, by at least about 50%, by at least about 60%, by at least about 70%, by at least about 80%, or by at least about 90%). The plurality of polymers can increase the stability of the solid dispersion (e.g., when stored at 4° C. or at room temperature) by at least about 10% (e.g., by at least about 20%, by at least about 30%, by at least about 40%, by at least about 50%, by at least about 60%, by at least about 70%, by at least about 80%, or by at least about 90%) as compared to a solid dispersion stored under identical conditions and in the absence of a polymer.

Further, without being bound by theory, the presence of a plurality of polymers can help prevent, decrease, or slow the amount or rate of crystallization of the drug as compared to the amount or rate of crystallization that occurs in the presence of one polymer. For example, the amount of crystallization when a plurality of polymers is used can be decreased by at least about 10%, by at least about 20%, by at least about 30%, by at least about 40%, by at least about 50%, by at least about 60%, by at least about 70%, by at least about 80%, by at least about 90%, by at least about 95%, or by at least about 99% compared to the amount of crystallization in the presence of one polymer. For example, a plurality of polymers can protect a drug against crystallization in an aqueous medium, such as gastric fluids or in intestinal fluids. For example, a plurality of polymers, e.g., a mixture comprising HPMC and HPMCAS, can offer increased protection to a given dispersion of VX-950: for example, the HMPC can protect the VX-950 from crystallization in gastric fluids or SGF while the HPMCAS can protect the VX-950 from crystallization in intestinal fluids or in SIF. As a result, use of a mixture can offer improved bioavailability, solubility, and/or absorption of VX-950. In addition, a plurality of polymers can increase the shelf stability of a composition, e.g., a solid form (e.g., a spray dried dispersion, a tablet), containing VX-950 relative to the stability of the composition when one polymer is used by at least about 10% (e.g., by at least about 20%, by at least about 30%, by at least about 40%, by at least about 50%, by at least about 60%, by at least about 70%, by at least about 80%, or by at least about 90%). The plurality of polymers can increase the stability of the solid dispersion (e.g., when stored at 4° C. or at room temperature) by at least about 10% (e.g., by at least about 20%, by at least about 30%, by at least about 40%, by at least about 50%, by at least about 60%, by at least about 70%, by at least about 80%, or by at least about 90%) as compared to a solid dispersion stored under identical conditions and containing one polymer.

The plurality of polymers (e.g., containing one or more cellulosic polymers) can be used to provide a form of VX-950 such that when administered, the area under curve (AUC) of VX-950 would be substantially the same in fasted and fed subjects, e.g., reducing or substantially eliminating the food effect in the subject.

In one embodiment, a plurality of polymers, or one or more of the polymers in a plurality of polymers of the present disclosure are able to dissolve in aqueous media. The solubility of the polymer(s) may be pH-independent or pH-dependent. The latter include one or more enteric polymers. The term “enteric polymer” refers to a polymer that is preferentially soluble in the less acidic environment of the intestine relative to the more acid environment of the stomach, for example, a polymer that is insoluble in acidic aqueous media but soluble when the pH is above 5-6. An appropriate polymers should be chemically and biologically inert. In order to improve the physical stability of the solid dispersions, the glass transition temperature (T_{g}) of the polymers (e.g., of a plurality of polymers, or one or more of the polymers in a plurality of polymers) should be as high as possible. For example, preferred polymers have a glass transition temperature at least equal to or greater than the glass transition temperature of the drug (e.g., VX-950). Other preferred polymers have a glass transition temperature that is within about 10 to about 15° C. of the drug (e.g., VX-950). Examples of suitable glass transition temperatures of the polymers include at least about 90° C., at least about 95° C., at least about 100° C., at least about 105° C., at least about 110° C., at least about 115° C., at least about 120° C., at least about 125° C., at least about 130° C., at least about 135° C., at least about 140° C., at least about 145° C., at least about 150° C., at least about 155° C., at least about 160° C., at least about 165° C., at least about 170° C., or at least about 175° C. (as measured under dry conditions). Without wishing to be bound by theory, it is believed that the underlying mechanism is that a polymer with a higher T_{g} generally has lower molecular mobility at room temperature, which can be a crucial factor in stabilizing the physical stability of the amorphous solid dispersion.

Additionally, the hygroscopicity of the polymer(s) (e.g., of a plurality of polymers, or one or more of the polymers in a plurality of polymers) should be as low as possible. For the purpose of comparison in this application, the hygroscopicity of a polymer, combination of polymers, or composition is characterized at about 60% relative humidity. In some preferred embodiments, the polymers have less than about 10% water absorption, for example less than about 9%, less than about 8%, less than about 7%, less than about 6%, less than about 5%, less than about 4%, less than about 3%, or less than about 2% water absorption. Cellulosic polymers generally have about 3% water absorption whereas PVP generally has about 9% water absorption. The hygroscopicity can also affect the physical stability of the solid dispersions. Generally, moisture adsorbed in the polymers can greatly reduce the T_{g} of the polymers as well as the resulting solid dispersions, which will further reduce the physical stability of the solid dispersions as described above.

In one embodiment, a plurality of polymers, or one or more of the polymers in a plurality of polymers is one or more water-soluble polymer(s) or partially water-soluble polymer(s). Water-soluble or partially water-soluble polymers include but are not limited to, cellulose derivatives (e.g., hydroxypropylmethylcellulose (HPMC), hydroxypropylcellulose (HPC)) or ethylcellulose; polyvinylpyrrolidones (PVP); polyethylene glycols (PEG); polyvinyl alcohols (PVA); acrylates, such as polymethacrylate (e.g., Eudragit® E); cyclodextrins (e.g., β-cyclodextin) and copolymers and derivatives thereof, including for example PVP-VA (polyvinylpyrollidone-vinyl acetate), PVP K30 (polyvinylpyrrolidone). In some preferred embodiments, one of the plurality of polymers is hydroxypropylmethylcellulose (HPMC), such as HPMC E50 (e.g., from Dow), HPMCE15, or HPMC 60SH 50 cP (e.g., Shin-Etsu Metolose, HPMC60SH50). HPMC is available in a variety of types from Shin-Etsu, including SM, 60SH, 65SH, 90SH. Each of these types vary by viscosity grade and methoxyl and hydroxypropoxyl content. A most preferred type for use in the spray dispersion is HPMC 60SH.

In some embodiments, a plurality of polymers, or one or more of the polymers in a plurality of polymers are a pH-dependent enteric polymer. Such pH-dependent enteric polymers include, but are not limited to, cellulose derivatives (e.g., cellulose acetate phthalate (CAP)), hydroxypropyl methyl cellulose phthalates (HPMCP), hydroxypropyl methyl cellulose acetate succinate (HPMCAS), carboxymethylcellulose (CMC) or a salt thereof (e.g., a sodium salt such as (CMC-Na)); cellulose acetate trimellitate (CAT), hydroxypropylcellulose acetate phthalate (HPCAP), hydroxypropylmethyl-cellulose acetate phthalate (HPMCAP), and methylcellulose acetate phthalate (MCAP), or polymethacrylates (e.g., Eudragit® S). In some preferred embodiments, one of the plurality of polymers is hydroxypropyl methyl cellulose acetate succinate (HPMCAS). HPMCAS is available in a variety of grades from Shin-Etsu, including AS-LF, AS-MF, AS-HF, AS-LG, AS-MG, AS-HG. Each of these grades vary with the percent substitution of acetate and succinate. A most preferred grade for use in the spray dispersion is AS-HG from Shin-Etsu.

In yet another embodiment, one or more of the polymers in a plurality of polymers is an insoluble cross-linked polymer, for example a polyvinylpyrrolidone (e.g., Crospovidone).

In embodiments where the drug forms a solid dispersion with a plurality of polymers, for example VX-950 with an HPMC and/or an HPMCAS polymer, the total amount of polymers relative to the total weight of the solid dispersion is typically at least about 5% (e.g., about 4% or 6%), at least about 10% (e.g., 9% or 11%), at least about 15% (e.g., 14% or 16%), at least about 20% (e.g., 19% or 21%), and preferably at least about 30% (e.g., about 29% or 31%), for example, at least about 35% (e.g., about 34% or 36%), at least about 40% (e.g., about 39% or 41%), at least about 45% (e.g., about 44% or 46%), or at least about 50% (e.g., about 49% or 51%). The amount is typically about 99% or less, and preferably about 80% or less, for example about 75% or less, about 70% or less, about 65% or less, about 60% or less, or about 55% or less. In one embodiment, the polymers are in an amount of up to about 30% of the total weight of the dispersion (and even more specifically, between about 28% and 32%, such as about 29%). In one embodiment, the polymers are in an amount of up to about 35% of the total weight of the dispersion (and even more specifically, between about 33% and 37%, such as about 34%). In one embodiment, the polymers are in an amount of up to about 40% of the total weight of the dispersion (and even more specifically, between about 38% and 42%, such as about 39%). In one embodiment, the polymers are in an amount of up to about 45% of the total weight of the dispersion (and even more specifically, between about 43% and 47%, such as about 44%).

The solid (e.g., spray dried) dispersions containing VX-950 can contain a plurality of polymers. For example, two polymers can be used in the dispersion. In some embodiments, the plurality of polymers can include one or more than one cellulosic polymer. For example, a spray dried dispersion can include two cellulosic polymers, e.g., HPMC and HPMCAS. In some embodiments, the solid dispersion includes a mixture of HPMC and HPMCAS. The amount of each polymer used in the dispersion can vary, and the ratio of the polymers to each other can also vary. For example, the dispersion can include from about 0% to about 100% by weight of a first polymer (e.g., HPMC) and from about 0% to about 100% by weight of a second polymer (e.g., HPMC AS) (wherein the percentages by weight of the two polymers add up to 100% of total polymer present in a dispersion). For example, in a solid dispersion of VX-950 containing polymers, the first polymer is present in an amount of about 33% and the second polymer is present in an amount of about 67% of the total amount of polymer added. In another example, the first polymer is present in an amount of about 55.5% and the second polymer is present in an amount of about 44.5% of the total amount of polymer added. In another example, the first polymer is present in an amount of about 63% and the second polymer is present in an amount of about 37% of the total amount of polymer added. In another example, the first polymer is present in an amount of about 50% and the second polymer is present in an amount of about 50% of the total amount of polymer added. In another example, the first polymer is present in an amount of about 100% and the second polymer is present in an amount of about 0% of the total amount of polymer added.

In one of the more specific embodiments of this disclosure, one of the polymers is polyvinylpyrrolidone (PVP) (e.g., PVP29/32). The PVP can be present in an amount of up to about 35%, up to about 40%, up to about 45%, or up to about 50%. A dispersion comprising about 50% (e.g., about 49.5%) PVP K29/32 is included within this disclosure.

In another embodiment, the disclosure includes a solid dispersion of VX-950 and at least two cellulosic polymers, for example an HPMC and/or an HPMCAS polymer. In some preferred embodiments, the drug (i.e., VX-950) is present in an amount of at least about 50% of the dispersion, for example at least about 55%, at least about 60%, at least about 65%, at least about 70%, at least about 75%, at least about 80%, at least about 85%, at least about 90%, or even greater. In some preferred embodiments, the drug is present in an amount between about 55% and about 70%, such as about 55%, about 60%, about 65%, or about 70%. As described above, the total amount of polymers is present in an amount of at least about 15%, and preferably at least about 20%, for example, at least about 25%, at least about 30%, at least about 35%, at least about 40%, or at least about 45%. In some embodiments, the amount is typically about 55% or less, and preferably about 50% or less, for example about 45% or less, about 40% or less, about 35% or less, about 30% or less, about 25% or less, about 20% or less, about 15% or less, or about 10% or less.

In some preferred embodiments, the dispersion further includes other minor ingredients, such as a surfactant (e.g., SLS or Vitamin E TPGS). In some preferred embodiments, the surfactant is present in less than about 10% by weight of the dispersion, for example less than about 9% by weight, less than about 8% by weight, less than about 7% by weight, less than about 6% by weight, less than about 5% by weight, less than about 4% by weight, less than about 3% by weight, less than about 2% by weight, or about 1% by weight.

The plurality of polymers should be present in an amount effective for stabilizing the solid dispersion. Stabilizing includes inhibiting or decreasing the crystallization of VX-950. Such stabilizing would inhibit the conversion VX-950 from amorphous to crystalline form. For example, the polymers would prevent at least a portion (e.g., about 5%, about 10%, about 15%, about 20%, about 25%, about 30%, about 35%, about 40%, about 45%, about 50%, about 55%, about 60%, about 65%, about 70%, about 75%, or greater) of VX-950 from going from an amorphous to a crystalline form.

For example, at low pH (e.g., in gastric fluid (e.g., fasted gastric fluid) or SGF (e.g., fasted SGF), VX-950 may dissolve, become supersaturated, and then crystallize. The plurality of polymers can prevent or decrease the crystallization of VX-950 in such or similar conditions, or during storage of a composition containing VX-950. Stabilization can be measured, for example, by measuring the glass transition temperature of the solid dispersion, measuring the rate of relaxation of the amorphous material, or by measuring the solubility or bioavailability of VX-950.

A plurality of polymers can be used in a formulation with VX-950. One, more than one, or all of the polymers suitable for use in combination with VX-950, for example to form a solid dispersion such as an amorphous solid dispersion, should have one or more of the following properties:

1. The glass transition temperature of the polymers in combination should have a temperature of no less than about 10-15° C. lower than the glass transition temperature of VX-950. Preferably, the glass transition temperature of the polymers in combination is greater than the glass transition temperature of VX-950, and in general at least 50° C. higher than the desired storage temperature of the drug product. For example, at least about 100° C., at least about 105° C., at least about 105° C., at least about 110° C., at least about 120° C., at least about 130° C., at least about 140° C., at least about 150° C., at least about 160° C., at least about 160° C., or greater.

2. The polymers in combination should be relatively non-hygroscopic. For example, the polymers should, when stored under standard conditions, absorb less than about 10% water, for example, less than about 9%, less than about 8%, less than about 7%, less than about 6%, or less than about 5%, less than about 4%, or less than about 3% water. Preferably the polymers will, when stored under standard conditions, be substantially free of absorbed water.

3. The polymers in combination should have similar or better solubility in solvents suitable for spray drying processes relative to that of VX-950. In preferred embodiments, the polymers will dissolve in one or more of the same solvents or solvent systems as VX-950. It is preferred that the polymers are soluble in at least one non-hydroxy containing solvent such as methylene chloride, acetone, or a combination thereof.

4. The polymers in combination, when combined with VX-950, for example in a solid dispersion, should increase the solubility of VX-950 in aqueous and physiologically relative media either relative to the solubility of VX-950 in the absence of polymers or relative to the solubility of VX-950 when combined with a reference polymer. For example, the polymers could increase the solubility of amorphous VX-950 by reducing the amount of amorphous VX-950 that converts to crystalline VX-950 from a solid amorphous dispersion.

5. The polymers in combination should decrease the relaxation rate of the amorphous substance.

6. The polymers in combination should increase the physical and/or chemical stability of VX-950.

7. The polymers in combination should improve the manufacturability of VX-950.

8. The polymers in combination should improve one or more of the handling, administration or storage properties of VX-950.

9. The polymers in combination should not interact unfavorably with other pharmaceutical components, for example excipients.

The suitability of candidate polymers (or other component) can be tested using the spray drying methods (or other methods) described herein to form an amorphous composition. The candidate composition can be compared in terms of stability, resistance to the formation of crystals, or other properties, and compared to a reference preparation, e.g., a preparation described herein, e.g., a preparation of about 55% amorphous VX-950, about 44% HPMC and/or HPMCAS (e.g., a mixture of 24.4% HPMC and 19.6% HPMC AS; percent is of total weight of the dispersion), and about 1% of a surfactant, e.g., SLS or vitamin E TPGS; or crystalline VX-950. E.g., a candidate composition could be tested to determine whether it inhibits the time to onset of solvent mediated crystallization, or the percent conversion at a given time under controlled conditions, by at least 50%, 75%, 100%, or 110% as well as the reference preparation, or a candidate composition could be tested to determine if it has improved bioavailability or solubility relative to crystalline VX-950.

An especially preferred embodiment includes a solid dispersion of VX-950, HPMC, HPMCAS, and a surfactant. For example a solid dispersion including about 55% VX-950, between about 15% and about 25% (e.g., about 19.6%) of an HPMC polymer, such as HPMC60SH50, between about 20% and about 30% (e.g., about 24.4%) of an HPMCAS polymer, such as HPMCAS-HG, and about 1% of a surfactant, such as SLS.

Another preferred embodiment includes a solid dispersion including about 55% VX-950, between about 25% and about 35% (e.g., about 29.3%) of an HPMC polymer, such as HPMC60SH50, between about 10% and about 20% (e.g., about 14.7%) of an HPMCAS polymer, such as HPMCAS-HG, and about 1% of a surfactant, such as SLS.

Another preferred embodiment includes a solid dispersion including about 60% VX-950, between about 10% and about 20% (e.g., about 14.6%) of an HPMC polymer, such as HPMC60SH50, between about 20% and about 30% (e.g., about 24.4%) of an HPMCAS polymer, such as HPMCAS-HG, and about 1% of a surfactant, such as SLS.

Another preferred embodiment includes a solid dispersion including about 65% VX-950, between about 12% and about 22% (e.g., about 17%) of an HPMC polymer, such as HPMC60SH50, between about 12% and about 22% (e.g., about 17%)of an HPMCAS polymer, such as HPMCAS-HG, and about 1% of a surfactant, such as SLS.

Another preferred embodiment includes a solid dispersion including about 70% VX-950, between about 15% and about 25% (e.g., about 19.3%) of an HPMC polymer, such as HPMC60SH50, between about 5% and about 15% (e.g., about 9.7%) of an HPMCAS polymer, such as HPMCAS-HG, and about 1% of a surfactant, such as SLS.

Surfactants

A solid dispersion, e.g., a spray-dried dispersion, or other composition may include a surfactant. A surfactant or surfactant mixture would generally decrease the interfacial tension between the solid dispersion and an aqueous medium. An appropriate surfactant or surfactant mixture may also enhance aqueous solubility and bioavailability of VX-950 from a solid dispersion. The surfactants for use in connection with the present disclosure include, but are not limited to, sorbitan fatty acid esters (e.g., Spans®), polyoxyethylene sorbitan fatty acid esters (e.g., Tweens®), sodium lauryl sulfate (SLS), sodium dodecylbenzene sulfonate (SDBS) dioctyl sodium sulfosuccinate (Docusate), dioxycholic acid sodium salt (DOSS), Sorbitan Monostearate, Sorbitan Tristearate, hexadecyltrimethyl ammonium bromide (HTAB), Sodium N-lauroylsarcosine, Sodium Oleate, Sodium Myristate, Sodium Stearate, Sodium Palmitate, Gelucire 44/14, ethylenediamine tetraacetic acid (EDTA), vitamin E or tocol derivates, such as alpha tocopherol, (e.g., d-alpha tocopherol, dl-alpha tocopherol, tocopherol succinate esters) and tocopheryl esters, such as tocopheryl acetate esters, tocopheryl succinate esters, e.g., Vitamin E d-alpha tocopheryl polyethylene glycol 1000 succinate (TPGS; e.g., Vitamin E TPGS from Eastman), Lecithin, MW 677-692, Glutanic acid monosodium monohydrate, Labrasol, PEG 8 caprylic/capric glycerides, Transcutol, diethylene glycol monoethyl ether, Solutol HS-15, polyethylene glycol/hydroxystearate, Taurocholic Acid, Pluronic F68, Pluronic F108, and Pluronic F127 (or any other polyoxyethylene-polyoxypropylene co-polymers (Pluronics®) or saturated polyglycolized glycerides (Gelucirs®)). Specific example of such surfactants that may be used in connection with this disclosure include, but are not limited to, Span 65, Span 25, Tween 20, Capryol 90, Pluronic F108, sodium lauryl sulfate (SLS), Vitamin E TPGS, pluronics and copolymers. SLS (e.g., Sigma or Fischer) and Vitamin E TPGS are preferred.

The amount of the surfactant (e.g., SLS or Vitamin E TPGS) relative to the total weight of the solid dispersion may be between about 0.1-20%. Preferably, it is from about 1% to about 20%, about 1 to about 15%, about 1 to about 10%, more preferably from about 1 to about 5%, e.g., about 1%, about 2%, about 3%, about 4%, or about 5%.

In certain embodiments, the amount of the surfactant relative to the total weight of the solid dispersion is at least about 0.1%, preferably at least about 0.5%, and more preferably at least about 1% (e.g., about 1%). In these embodiments, the surfactant would be present in an amount of no more than about 20%, and preferably no more than about 15%, about 12%, about 11%, about 10%, about 9%, about 8%, about 7%, about 6%, about 5%, about 4%, about 3%, about 2% or about 1%. As shown in the examples herein, an embodiment wherein the surfactant is in an amount of about 1% by weight is preferred.

Candidate surfactants (or other components) can be tested for suitability for use in the disclosure in a manner similar to that described for testing polymers.

Compositions/Packaging/Use

Pharmaceutical compositions are also provided herein. The forms of VX-950 and the solid dispersions according to this disclosure may be further processed for preparing a pharmaceutical composition for administering to a patient. Although a solid dispersion could be considered a pharmaceutical composition, further processing may be needed prior to administration (for example, the solid dispersion may be further formulated into a tablet). All such pharmaceutical compositions, dosage forms, and pharmaceutical formulations would be included within this disclosure (e.g., sustained release or immediate release formulations). The formulations may be prepared using known components according to known methods (see, Handbook of Pharmaceutical Excipients). As would be appreciated, oral formulations are often preferred for pharmaceutical administration.

Accordingly, a pharmaceutical composition comprising VX-950 is provided herein. Such compositions typically contain a pharmaceutically acceptable carrier, diluent, or vehicle. In some embodiments, the VX-950 is in amorphous form. In some embodiments, the VX-950 is in the form of a solid dispersion (e.g., an amorphous solid dispersion). These VX-950 forms and dispersions are preferably prepared as disclosed herein.

The compositions and processes of this disclosure may optionally include one or more excipients (see U.S. Pat. No. 6,720,003, US 2004/0030151, and/or WO 99/02542)). An excipient is a substance used as a carrier or vehicle in a dosage form, or added to a pharmaceutical composition, to improve handling, storage, or preparation of a dosage form. Excipients include, but are not limited to, diluents, disintegrants, adhesives, wetting agents, lubricants, glidants, crystallization inhibitors, surface modifying agents, agents to mask or counteract a disagreeable taste or odor, flavors, dyes, fragrances, fillers, binders, stabilizers and substances to improve the appearance of a composition.

Processes for preparing a formulation comprising an amorphous form of VX-950, or a dispersion or composition thereof, into a dosage form suitable to administration to a mammal are also included herein. Preferably, the formulation comprises a solid dispersion prepared as described herein.

Accordingly, another embodiment of this disclosure provides a composition comprising VX-950, or a pharmaceutically acceptable salt thereof. According to a preferred embodiment, VX-950 is present in an amount effective to decrease the viral load in a sample or in a patient (e.g., decrease the plasma level of the virus at least about 3 log, at least about 4 log, or at least about 5 log), and a pharmaceutically acceptable carrier. Alternatively, a composition of this disclosure comprises another additional agent as described herein (e.g., a CYP inhibitor). Each component may be present in individual compositions, combination compositions, or in a single composition.

As used herein the term “comprising” is intended to be open-ended, thus indicating the potential inclusion of other agents in addition to the specified agents.

As used herein, the compounds of this disclosure, including VX-950, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this disclosure (for example an imidate ester of an amide), which, upon administration to a recipient, is capable of providing (directly or indirectly) a compound of this disclosure. Particularly favored derivatives and prodrugs are those that increase the bioavailability of the compounds of this disclosure when such compounds are administered to a mammal (e.g., by allowing an orally administered compound to be more readily absorbed into the blood) or which enhance delivery of the parent compound to a biological compartment (e.g., the liver, brain or lymphatic system) relative to the parent species. Preferred prodrugs include derivatives where a group which enhances aqueous solubility or active transport through the gut membrane is appended to the structure of formulae described herein.

The VX-950 utilized in the compositions and methods of this disclosure may also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose based substances, polyethylene glycol, sodium carboxymethylcellulose, polyacrylates, waxes, polyethylene polyoxypropylene block polymers, polyethylene glycol and wool fat.

The pharmaceutical compositions of this disclosure may be orally administered in any orally acceptable dosage form including, but not limited to, capsules, tablets, pills, powders, granules, aqueous suspensions or solutions. In the case of tablets for oral use, carriers that are commonly used include lactose, microcrystalline cellulose, mannitol, dicalcium phosphate, calcium carbonate and corn starch. Lubricating agents, such as magnesium stearate, sodium stearyl fumerate, or stearic acid, are also typically added. Other ingredients may include disintegrants, such as crosscarmellose sodium or sodium starch glycolate, flow aids such as colloidal silica, and surfactants, such as SLS and Vitamin E, may be included. For oral administration in a capsule form, useful diluents include lactose, microcrystalline cellulose, mannitol, dicalcium phosphate, calcium carbonate and dried cornstarch. Similar to the tablet formulations described above, capsule formulations may also contain lubricants, disintegrants, surfactants, or flow aids. In some embodiments a tablet is coated with a film, e.g., to increase ease of swallowing. Also, an enteric coating can be applied to the oral dosage form to control where the composition is absorbed in the digestive system (e.g., the coating can dissolve in the higher pH of the small intestine but not in the acidic pH of the stomach). Examples of enteric coatings include: methacrylic acid copolymers cellulose acetate (and its succinate and phthalate versions) styrol maleic acid co-polymers, polymethacrylic acid/acrylic acid copolymer, hydroxypropyl methyl cellulose phthalate, polyvinyl acetate phthalate, hydroxyethyl ethyl cellulose phthalate, hydroxypropyl methyl cellulose acetate succinate, cellulose acetate tetrahydrophtalate, acrylic resin, timellitate, and shellac. When aqueous suspensions are required for oral use, the active ingredient is combined with emulsifying and suspending agents. If desired, certain sweetening, flavoring or coloring agents may also be added. Acceptable liquid dosage forms include emulsions, solutions, suspensions, syrups, and elixirs.

According to a preferred embodiment, the compositions of this disclosure are formulated for pharmaceutical administration to a mammal, preferably a human being. Although the forms of VX-950 and the dispersions provided herein are preferably formulated for oral administration, other formulations could be obtained.

Other pharmaceutical compositions of the present disclosure (as well as compositions for use in methods, combinations, kits, and packs of this disclosure) may be administered orally, parenterally, sublingually, by inhalation spray, topically, rectally, nasally, buccally, vaginally or via an implanted reservoir. The term “parenteral” as used herein includes subcutaneous, intravenous, intramuscular, intra articular, intra synovial, intrasternal, intrathecal, intrahepatic, intralesional and intracranial injection or infusion techniques. Preferably, the compositions are administered orally or intravenously.

The disclosure also provides pharmaceutical packs and kits comprising amorphous VX-950, a solid dispersion, or a pharmaceutical composition according to any of the embodiments herein.

The disclosure further provides methods for treating or preventing hepatitis C virus infection in a patient comprising administering to the patient a pharmaceutical composition. The pharmaceutical composition comprises any form of VX-950, any solid dispersion, or any composition according to this disclosure.

According to another embodiment, the disclosure provides a method for treating a patient infected with a virus, e.g., an HCV, characterized by a virally encoded NS3/4A serine protease that is necessary for the life cycle of the virus by administering to said patient any form of VX-950, any solid dispersion, or a composition according of this disclosure. Preferably, methods of this disclosure are used to treat a patient suffering from a HCV infection. Such treatment may completely eradicate the viral infection or reduce the severity thereof. More preferably, the patient is a human being.

In yet another embodiment, the present disclosure provides a method of pre-treating a biological substance intended for administration to a patient comprising the step of contacting said biological substance with a pharmaceutically acceptable composition comprising a compound of this disclosure. Such biological substances include, but are not limited to, blood and components thereof such as plasma, platelets, subpopulations of blood cells and the like; organs such as kidney, liver, heart, lung, etc; sperm and ova; bone marrow and components thereof, and other fluids to be infused into a patient such as saline, dextrose, etc. In some embodiments, VX-950 can be placed on or in a device which is inserted into a patient.

Pharmaceutical compositions may also be prescribed to the patient in “patient packs” containing more than one dose, and preferably the whole course of treatment, in a single package, (e.g., a blister pack). Patient packs have an advantage over traditional prescriptions, where a pharmacist divides a patients supply of a pharmaceutical from a bulk supply, in that the patient always has access to the package insert contained in the patient pack, normally missing in traditional prescriptions. The inclusion of a package insert has been shown to improve patient compliance with the physician's instructions. Preferably the drug is in an oral dosage form.

It will be understood that the administration of the combination of the disclosure by means of a single patient pack, or patient packs of each formulation, containing within a package insert instructing the patient to the correct use of the disclosure is a desirable additional feature of this disclosure.

According to a further aspect of the disclosure is a pack comprising at least any form of VX-950, any solid dispersion, or any composition according to this disclosure and an information insert containing directions on the use of the combination of the disclosure. In an alternative embodiment of this disclosure, the pharmaceutical pack further comprises one or more of additional agents as described herein. The additional agent or agents may be provided in the same pack or in separate packs.

Another aspect of this involves a packaged kit for inhibiting HCV, or for a patient to use in the treatment of HCV infection or in the prevention of HCV infection, comprising: a single or a plurality of pharmaceutical formulation of each pharmaceutical component; a container housing the pharmaceutical formulation(s) during storage and prior to administration; and instructions for carrying out drug administration in a manner effective to treat or prevent HCV infection. Preferably, the drug is in an oral dosage form.

Accordingly, this disclosure provides kits for the simultaneous or sequential administration of VX-950 (and optionally an additional agent) or derivatives thereof are prepared in a conventional manner. Typically, such a kit will comprise, e.g., a composition of each inhibitor and optionally the additional agent(s) in a pharmaceutically acceptable carrier (and in one or in a plurality of pharmaceutical formulations) and written instructions for the simultaneous or sequential administration. Preferably the drug is in an oral dosage form.

In another embodiment, a packaged kit is provided that contains one or more dosage forms (preferably an oral dosage form) for self administration; a container means, preferably sealed, for housing the dosage forms during storage and prior to use; and instructions for a patient to carry out drug administration. The instructions will typically be written instructions on a package insert, a label, and/or on other components of the kit, and the dosage form or forms are as described herein. Each dosage form may be individually housed, as in a sheet of a metal foil-plastic laminate with each dosage form isolated from the others in individual cells or bubbles, or the dosage forms may be housed in a single container, as in a plastic bottle or a vial. The present kits will also typically include means for packaging the individual kit components, i.e., the dosage forms, the container means, and the written instructions for use. Such packaging means may take the form of a cardboard or paper box, a plastic or foil pouch, etc.

Embodiments of this disclosure may also involve additional agents. Therefore, a method of this disclosure may involve steps as administering such additional agents.

Dosage

Dosage levels of from about 0.01 to about 100 mg/kg body weight per day, preferably from about 10 to about 100 mg/kg body weight per day of VX-950 are useful for the prevention and treatment of HCV mediated disease. In some embodiments, dosage levels are from about 0.4 to about 10 g/day, for example from about 1 to about 4 g/day, preferably from about 2 to about 3.5 g/day, per person (based on the average size of a person calculated at about 70 kg) are included. Typically, the pharmaceutical compositions of, and according to, this invention will be administered from about 1 to about 5 times per day, preferably from about 1 to about 3 times per day, or alternatively, as a continuous infusion. In some embodiments, VX-950 is administered using a controlled release formulation. In some embodiments, this can help to provide relatively stable blood levels of VX-950.

In some embodiments, the dose of amorphous VX-950 can be a standard dose, e.g., about 1 g to about 5 g a day, more preferably about 2 g to about 4 g a day, more preferably about 2 g to about 3 g a day, e.g., about 2.25 g or about 2.5 g a day. For example, a dose of about 2.25 g/day of amorphous VX-950 can be administered to a patient, e.g., about 750 mg administered three times a day. Such a dose can be administered, e.g., as three 250 mg doses three times a day or as two 375 mg doses three times a day. In some embodiments, the 250 mg dose is in an about 700 mg tablet. In some embodiments, the 375 mg dose is in an about 800 mg tablet. As another example, a dose of about 2.5 g/day of amorphous VX-950 can be administered to a patient, e.g., about 1250 mg administered two times a day. As another example, about 1 g to about 2 g of amorphous VX-950 a day can be administered to a patient, e.g., about 1.35 g of amorphous VX-950 can be administered to a patient, e.g., about 450 mg administered three times a day. The dose of amorphous VX-950 can be administered e.g., as a spray dried dispersion or as a tablet (e.g., a tablet that comprises VX-950, e.g., in a spray dried dispersion).

In some embodiments, the solid (e.g., spray dried) dispersions of VX-950 described herein contain at least about 50%, at least about 55%, at least about 60%, at least about 65%, at least about 70%, at least about 75%, at least about 80%, at least about 85% or greater of VX-950 (e.g., amorphous VX-950). Because these dispersions can include greater amounts of VX-950 for a given amount of a dispersion (e.g., a greater percent by weight of VX-950), for the same amount by weight of solid dispersion, a greater amount of VX-950 can be incorporated into a pharmaceutical composition, thereby increasing the load of the active ingredient in that composition. As a result, a subject receiving VX-950 can take fewer doses of VX-950 and yet intake the same amount of drug. For example, to receive a dose of 750 mg of VX-950, a subject can take two 375 mg doses of VX-950 containing a solid dispersion described herein instead of three 250 mg doses. This can be an improvement or a preferred dose for some patients. As another example, the increased load of amorphous VX-950 in a solid dispersion can allow administration of a larger dose of VX-950 to a subject in a fixed total dose of a pharmaceutical composition (e.g., a tablet of a standard size may contain a larger percentage (and thereby dose) of amorphous VX-950). Conversely, the increased load of amorphous VX-950 can allow a fixed dose amount of amorphous to be administered to a subject in a small total dose of a pharmaceutical composition (e.g., a standard dose of amorphous VX-950 can be administered in a smaller tablet).

In some embodiments, the amorphous VX-950 is not 100% potent or pure (e.g., the potency or purity is at least about 90%, at least about 92%, at least about 93%, at least about 94%, at least about 95%, at least about 96%, at least about 97%, at least about 98%, or at least about 99% potent), in which case the doses described above refer to the amount of potent or pure VX-950 administered to a patient rather than the total amount of VX-950. These doses can be administered to a patient as a monotherapy and/or as part of a combination therapy, e.g., as described further below.

Such administration can be used as a chronic or acute therapy. The amount of active ingredient that may be combined with the carrier materials to produce a single dosage form will vary depending upon the subject treated and the particular mode of administration. A typical preparation will contain from about 5% to about 95% active compound (w/w). Preferably, such preparations contain from about 20% to about 80%, from about 25% to about 70%, from about 30% to about 60% active compound.

When the compositions or methods of this disclosure involve a combination of VX-950 and one or more additional therapeutic or prophylactic agents, both the compound and the additional agent should be present at dosage levels of between about 10 to 100%, and more preferably between about 10 to 80% of the dosage normally administered in a monotherapy regimen.

Upon improvement of a patient's condition, a maintenance dose of a compound, composition or combination of this disclosure may be administered, if necessary. Subsequently, the dosage or frequency of administration, or both, may be reduced, e.g., to about ½ or ¼ or less of the dosage or frequency of administration, as a function of the symptoms, to a level at which the improved condition is retained when the symptoms have been alleviated to the desired level, treatment should cease. Patients may, however, require intermittent treatment on a long-term basis upon any recurrence of disease symptoms.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredients will also depend upon the particular described compound and the presence or absence and the nature of the additional anti-viral agent in the composition.

Combination Therapy

Methods of this disclosure may also involve administration of another component comprising an additional agent selected from an immunomodulatory agent; an antiviral agent; an inhibitor of HCV protease; an inhibitor of another target in the HCV life cycle; an inhibitor of internal ribosome entry, a broad-spectrum viral inhibitor; another cytochrome P-450 inhibitor; or combinations thereof.

Accordingly, in another embodiment, this invention provides a method comprising administering any form of VX-950, any solid dispersion, or any composition according to this disclosure, a CYP inhibitor, and another anti-viral agent, preferably an anti-HCV agent. Such anti-viral agents include, but are not limited to, immunomodulatory agents, such as α-, β-, and γ-interferons, pegylated derivatized interferon-α compounds, and thymosin; other anti-viral agents, such as ribavirin, amantadine, and telbivudine; other inhibitors of hepatitis C proteases (NS2-NS3 inhibitors and NS3/NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds of U.S. Pat. Nos. 5,807,876, 6,498,178, 6,344,465, 6,054,472; International Applications WO 97/40028, WO 98/40381, WO 00/56331, and mycophenolic acid and derivatives thereof, and including, but not limited to VX-497, VX-148, and/or VX-944); or combinations of any of the above.

A preferred combination therapy comprises a dose of amorphous VX-950 described herein and interferon-α, e.g., pegylated derivatized interferon-α (e.g., pegylated interferon-alpha-2a; e.g., PEGASYS®, e.g., at its standard dose, or pegylated interferon-alpha-2b, e.g., PEG-INTRON® (e.g., REDIPEN PEG-INTRON®), e.g., at its standard dose). For example, a dose (e.g., as described above) of amorphous VX-950, e.g., about 2 g to about 3 g (e.g., 2.5 g, 2.25 g (e.g., 750 mg three times a day)), e.g., in the form described herein can be administered three times a day and pegylated interferon-alpha-2a can be administered at a standard dose, e.g., 180 μg once weekly by subcutaneous administration, e.g., for 48 weeks. As another example, a dose of VX-950 can be administered with both pegylated interferon-alpha-2 and ribavirin. For example, about 2 g to about 3 g (e.g., about 2.5 g, about 2.25 g (e.g., 750 mg three times a day)) of amorphous VX-950 described herein, can be administered three times a day in combination with 180 μg of pegylated interferon-alpha-2a (e.g., PEGASYS®) once a week and ribavirin (e.g., COPEGUS®, REBETOL®) at 1000-1200 mg/day, e.g., for 48 weeks, for genotype 1 patients, or in combination with 180 μg of pegylated interferon-alpha-2a once a week plus ribavirin at 800 mg/day for patients with genotype 2 or 3 hepatitis C.

Each agent may be formulated in separate dosage forms. Alternatively, to decrease the number of dosage forms administered to a patient, each agent may be formulated together in any combination. For example, the VX-950 may be formulated in one dosage form and any additional agents may be formulated together or in another dosage form. VX-950 can be dosed, for example, before, after or during the dosage of the additional agent.

A method according to this disclosure may also comprise the step of administering a cytochrome P450 monooxygenase inhibitor. CYP inhibitors may be useful in increasing liver concentrations and/or increasing blood levels of compounds (e.g., VX-950) that are inhibited by CYP.

The advantages of improving the pharmacokinetics of a drug (e.g., by administering a CYP inhibitor) are well accepted in the art. By administering a CYP inhibitor, this disclosure provides for decreased metabolism of the protease inhibitor, VX-950. The pharmacokinetics of the protease inhibitor are thereby improved. The advantages of improving the pharmacokinetics of a drug are well accepted in the art. Such improvement may lead to increased blood levels of the protease inhibitor. More importantly for HCV therapies, the improvement may lead to increased concentrations of the protease inhibitor in the liver.

In a method of this disclosure, the amount of CYP inhibitor administered is sufficient to increase the blood levels of the VX-950 as compared to the blood levels of this protease inhibitor in the absence of a CYP inhibitor. Advantageously, in a method of this disclosure, an even further lower dose of protease inhibitor may be therefore used (relative to administration of a protease inhibitor alone).

Accordingly, another embodiment of this disclosure provides a method for increasing blood levels or increasing liver concentrations of VX-950 in a patient receiving VX-950 comprising administering to the patient a therapeutically effective amount of VX-950 and a cytochrome P450 monooxygenase inhibitor.

In addition to treating patients infected with hepatitis C, the methods of this disclosure may be used to prevent a patient from becoming infected with hepatitis C. Accordingly, one embodiment of this disclosure provides a method for preventing a hepatitis C virus infection in a patient comprising administering to the patient a) any form of VX-950, any solid dispersion, or any composition according to this disclosure; and b) a cytochrome P450 monooxygenase inhibitor.

As would be realized by skilled practitioners, if a method of this disclosure is being used to treat a patient prophylactically, and that patient becomes infected with hepatitis C virus, the method may then treat the infection. Therefore, one embodiment of this disclosure provides any form of VX-950, any solid dispersion, or any composition according to this disclosure and a cytochrome P450 monooxygenase inhibitor wherein the combination of inhibitors are in therapeutically effective amounts for treating or preventing a Hepatitis C infection in a patient.

If an embodiment of this disclosure involves a CYP inhibitor, any CYP inhibitor that improves the pharmacokinetics of VX-950 may be used in a method of this disclosure. These CYP inhibitors include, but are not limited to, ritonavir (International Application WO 94/14436), ketoconazole, troleandomycin, 4-methyl pyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944 and VX-497. Preferred CYP inhibitors include ritonavir, ketoconazole, troleandomycin, 4-methyl pyrazole, cyclosporin, and clomethiazole. For preferred dosage forms of ritonavir, see U.S. Pat. No. 6,037, 157, and the documents cited therein: U.S. Pat. No. 5,484,801, U.S. application Ser. No. 08/402,690, and International Applications WO 95/07696 and WO 95/09614).

The structure of VX-944 is provided below.

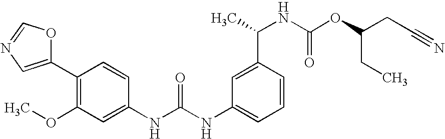

VX-497 is an IMPDH inhibitor. A combination of VX-497, pegylated IFN-α, and ribavirin is currently in clinical development for treating HCV [W. Markland et al., Antimicrobial & Antiviral Chemotherapy, 44, p. 859 (2000); U.S. Pat. N o. 6,541,496].

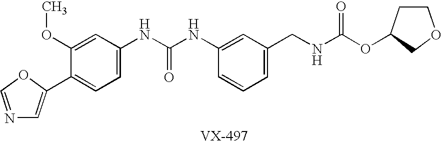

Methods for measuring the ability of a compound to inhibit cytochrome P50 monooxygenase activity are known (see U.S. Pat. No. 6,037,157 and Yun, et al. Drug Metabolism & Disposition, vol. 21, pp. 403-407 (1993).

A CYP inhibitor employed in this disclosure may be an inhibitor of only one isozyme or more than one isozyme. If the CYP inhibitor inhibits more than one isozyme, the inhibitor may nevertheless inhibit one isozyme more selectively than another isozyme. Any such CYP inhibitors may be used in a method of this disclosure.

In a method of this disclosure, the CYP inhibitor may be administered together with any form of VX-950, any solid dispersion, or any composition according to this disclosure in the same dosage form or in separate dosage forms.

If the CYP inhibitor and the other components of the combination are administered in separate dosage forms, each inhibitor may be administered about simultaneously. Alternatively, the CYP inhibitor may be administered in any time period around administration of the combination. That is, the CYP inhibitor may be administered prior to, together with, or following each component of the combination. The time period of administration should be such that the CYP inhibitor affects the metabolism of a component of the combination, preferably, of VX-950. For example, if VX-950 is administered first, the CYP inhibitor should be administered before VX-950 is substantially metabolized and/or excreted (e.g., within the half-life of VX-950).

In order that this disclosure be more fully understood, the following examples are set forth. These examples are for the purpose of illustration only and are not to be construed as limiting the scope of the disclosure in any way.

### Examples

VX-950 may be prepared in general by methods known to those skilled in the art (see, e.g., International Application WO 02/18369). HCV inhibition may be tested in HCV assays according to known methods

For the solid dispersion formulations presented in Examples 1-6, VX-950 and various amounts of HPMCAS-HG (Hypromellose Acetate Succinate, HG grade, Shin-Etsu Chemical Co.) polymer, HPMC-60SH50 (Metolose, Shin-Etsu Chemical Co.) polymer and SLS (Sodium Lauryl Sulfate, Sigma/Fisher) surfactant were used. Spray drying and subsequent post-drying in a qualified vacuum dryer were be performed. Success criteria included manufacturing the batches with reasonable yield (>60%), low residual solvents (<400 ppm for all OVIs) and matching target powder properties (primarily particle size and bulk/tap density), as well as meeting specifications for assay and purity.

For Examples 1-6, an 80/20 wt/wt mixture of methylene chloride and acetone was used and formulations were manufactured at about 10 wt% total solids concentration.

### Example 1

A solid dispersion was prepared comprising the following ingredients:

TABLE 1
Formulation composition: 55/24.4/19.6/1 w/w/w/w VX-
950/HPMCAS-HG/HPMC-60SH/SLS) per 1.250 kg of VX-950
(22.727 kg total batch size; 2.273 kg total solids).

| Dispersion | | |
|---|---|---|
| Component | | |
| Function | Dispersion Component | kg |
| API | VX-950 | 1.250 |
| Polymer/ | Hydroxypropyl Methylcellulose Acetate | 0.555 |
| Dispersant I | Succinate, JPE (Biddle Sawyer or Shin-Etsu | |
| | HPMCAS-HG grade) | |
| Polymer/ | Hydroxypropyl Methylcellulose 60SH 50 cP | 0.445 |
| Dispersant II | (Biddle Sawyer or Shin-Etsu Metolose, | |
| | HPMC60SH50) | |
| Surfactant | Sodium Lauryl Sulfate (SLS) | 0.023 |
| Process Solvent | Methylene Chloride, NF | 16.363 |
| | (for Dispersion) | |
| Process Solvent | Acetone, NF | 4.091 |
| | (for Dispersion) | |

### Example 2

A solid dispersion was prepared comprising the following ingredients:

TABLE 2
Formulation composition: 55/14.7/29.3/1 w/w/w/w VX-
950/HPMCAS-HG/HPMC-60SH/SLS) per 1.250 kg of VX-950
(22.727 kg total batch size; 2.273 kg total solids).

| Dispersion | | |
|---|---|---|
| Component | | |
| Function | Dispersion Component | kg |
| API | VX-950 | 1.250 |
| Polymer/ | Hydroxypropyl Methylcellulose Acetate | 0.334 |
| Dispersant I | Succinate, JPE (Biddle Sawyer or Shin-Etsu | |
| | HPMCAS-HG grade) | |
| Polymer/ | Hydroxypropyl Methylcellulose 60SH 50 cP | 0.666 |
| Dispersant II | (Biddle Sawyer or Shin-Etsu Metolose, | |
| | HPMC60SH50) | |
| Surfactant | Sodium Lauryl Sulfate (SLS) | 0.023 |
| Process Solvent | Methylene Chloride, NF | 16.363 |
| | (for Dispersion) | |
| Process Solvent | Acetone, NF | 4.091 |
| | (for Dispersion) | |

### Example 3

A solid dispersion was prepared comprising the following ingredients:

TABLE 3
Formulation composition: 60/24.4/14.6/1 w/w/w/w VX-
950/HPMCAS-HG/HPMC-60SH/SLS per 1.250 kg of VX-950
(20.83 kg total batch size; 2.083 kg total solids).

| Dispersion | | |
|---|---|---|
| Component | | |
| Function | Dispersion Component | kg |
| API | VX-950 | 1.250 |
| Polymer/ | Hydroxypropyl Methylcellulose Acetate | 0.508 |
| Dispersant I | Succinate, JPE (Biddle Sawyer or Shin-Etsu | |
| | HPMCAS-HG grade) | |
| Polymer/ | Hydroxypropyl Methylcellulose 60SH 50 cP | 0.304 |
| Dispersant II | (Biddle Sawyer or Shin-Etsu Metolose, | |
| | HPMC60SH50) | |
| Surfactant | Sodium Lauryl Sulfate (SLS) | 0.021 |
| Process Solvent | Methylene Chloride, NF | 15.000 |
| | (for Dispersion) | |
| Process Solvent | Acetone, NF | 3.750 |
| | (for Dispersion) | |

### Example 4

A solid dispersion was prepared comprising the following ingredients:

TABLE 4
Formulation composition: 65/17/17/1 w/w/w/w VX-950/HPMCAS-
HG/HPMC-60SH/SLS per 1.250 kg of VX-950 (19.23 kg
total batch size; 1.923 kg total solids).

| Dispersion | | |
|---|---|---|
| Component | | |
| Function | Dispersion Component | kg |
| API | VX-950 | 1.250 |
| Polymer/ | Hydroxypropyl Methylcellulose Acetate | 0.327 |
| Dispersant I | Succinate, JPE (Biddle Sawyer or Shin-Etsu | |
| | HPMCAS-HG grade) | |
| Polymer/ | Hydroxypropyl Methylcellulose 60SH 50 cP | 0.327 |
| Dispersant II | (Biddle Sawyer or Shin-Etsu Metolose, | |
| | HPMC60SH50) | |
| Surfactant | Sodium Lauryl Sulfate (SLS) | 0.019 |
| Process Solvent | Methylene Chloride, NF | 13.846 |
| | (for Dispersion) | |
| Process Solvent | Acetone, NF | 3.462 |
| | (for Dispersion) | |

### Example 5

A solid dispersion was prepared comprising the following ingredients:

TABLE 5
Formulation composition: (70/9.7/19.3/1 w/w/w/w VX-
950/HPMCAS-HG/HPMC-60SH/SLS) per 1.250 kg of VX-950
(17.86 kg total batch size; 1.786 kg total solids).

| Dispersion | | |
|---|---|---|
| Component | | |
| Function | Dispersion Component | kg |
| API | VX-950 | 1.250 |
| Polymer/ | Hydroxypropyl Methylcellulose Acetate | 0.173 |
| Dispersant I | Succinate, JPE (Biddle Sawyer or Shin- | |
| | Etsu HPMCAS-HG grade) | |
| Polymer/ | Hydroxypropyl Methylcellulose 60SH 50 cP | 0.345 |
| Dispersant II | (Biddle Sawyer or Shin-Etsu Metolose, | |
| | HPMC60SH50) | |
| Surfactant | Sodium Lauryl Sulfate (SLS) | 0.018 |
| Process Solvent | Methylene Chloride, NF | 12.857 |
| | (for Dispersion) | |
| Process Solvent | Acetone, NF | 3.214 |
| | (for Dispersion) | |

### Example 6

A solid dispersion was prepared comprising the following ingredients:

TABLE 6
Formulation composition: (60/39/0/1 w/w/w/w VX-950/HPMCAS-
HG/HPMC-60SH/SLS) per 1.250 kg of VX-950 (20.833
kg total batch size; 2.083 kg total solids).

| Dispersion | | |
|---|---|---|
| Component | | |
| Function | Dispersion Component | kg |
| API | VX-950 | 1.250 |
| Polymer/ | Hydroxypropyl Methylcellulose Acetate | 0.813 |
| Dispersant I | Succinate, JPE (Biddle Sawyer or Shin- | |
| | Etsu HPMCAS-HG grade) | |
| Polymer/ | Hydroxypropyl Methylcellulose 60SH 50 cP | 0.000 |
| Dispersant II | (Biddle Sawyer or Shin-Etsu Metolose, | |
| | HPMC60SH50) | |
| Surfactant | Sodium Lauryl Sulfate (SLS) | 0.021 |
| Process Solvent | Methylene Chloride, NF | 15.000 |
| | (for Dispersion) | |
| Process Solvent | Acetone, NF | 3.750 |
| | (for Dispersion) | |

### Example 7

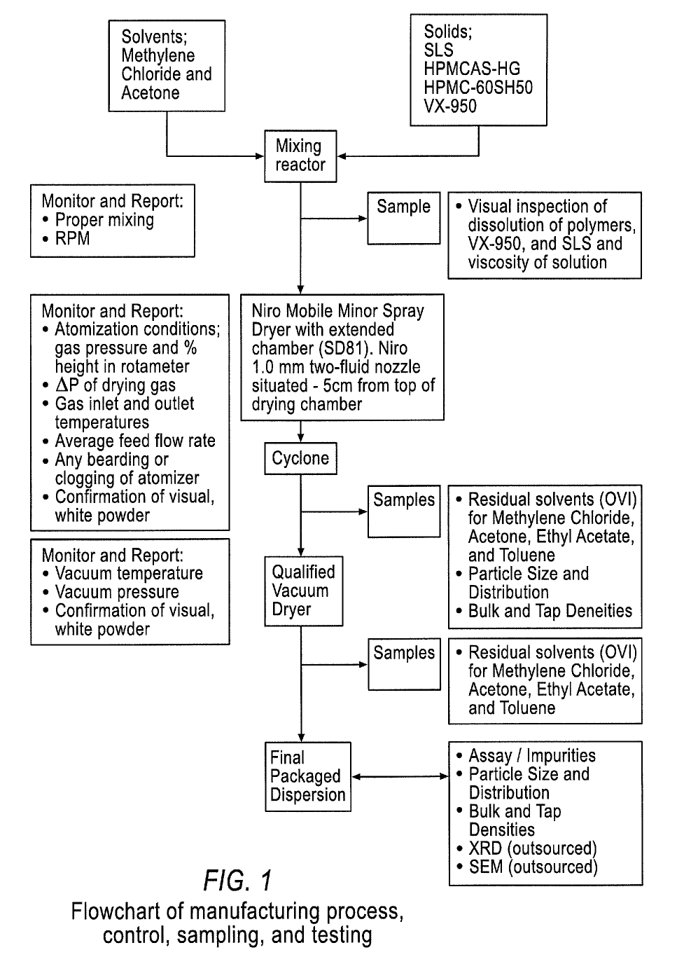

A flowchart schematic of the manufacturing process is given in FIG. 1.

The process flow performed for the dispersions described in Example 1-6 was as follows:

A) Preparation of Solution and Spray Dryer

1) Methylene Chloride was prepared in the equilibration solvent tank.

2) Acetone at the appropriate amount was prepared in the solution reactor. Calibrated scales confirmed the correct amount of charged solvent.

3) SLS was charged into the solution reactor and solubilized. Calibrated scales confirmed the correct amount of charged solid.

4) Methylene chloride at the appropriate amount was prepared in the solution reactor. Calibrated scales confirmed the correct amount of charged solvent.

5) The remaining solids (HPMCAS-HG, HPMC-60SH50, and VX-950) were charged into the solution reactor in the order listed solubilizing one at a time. The solids were mixed to 10 wt % to the mixture of methylene chloride (dichloromethane) and acetone (80/20 w/w). The resultant batch was tested for visual appearance and viscosity once dissolved.

6) The Niro 1.0 mm two-fluid nozzle was installed at approximately 5 cm from the top of the spray drying vessel and tested for correct atomization with the equilibration solvent.

B) Start-Up of the Spray Dryer

1) The spray dryer was heated to the appropriate outlet temperature. Operators confirmed that the collecting pots were dried.

2) Equilibration solvents were sprayed until all parameters were equilibrated and constant.

3) Spray drying of the feed solution was commenced once the spray dryer was equilibrated.

4) Dry particles were inertially separated from the process gas by a cyclone and collected within polyethylene bags. The process gas was then filtered for fine particles and condensed to remove process solvents.

5) An initial sample was taken and tested for particle size distribution and bulk and tap densities as well as GC for methylene chloride, acetone, ethyl acetate and toluene.

a) If particle size distribution and densities were within acceptance criteria and near targets, the process continued and samples were taken per the sampling plan.

b) If particle size distribution and densities were not within acceptance criteria and not near targets, the process was be optimized (by changing one or more of the following: outlet temperature, feed or atomization rate) as needed. Once the sample was within specification, the process was started with current parameters.

C) On-Going Spray Drying Process

1) Samples were taken per sampling plan.

2) Any changes to the processing parameters were noted.

3) Any stoppages or out of continuous operation occurrences were noted.

4) Upon completion of spray drying the feed solution, equilibration solvent was switched and normal shut down procedures were followed.

D) Post-Drying Process

1) Spray dried dispersion was charged into the qualified vacuum dryer. Samples were be taken per the sampling plan.

2) This post-drying continued until all residual solvents (e.g., methylene chloride, acetone) were below the specifications established.

E) Testing, Packaging, Shipment

1) Samples of this dispersion were tested for residual solvents (e.g., methylene chloride, acetone), particle size and distribution, bulk and tap densities, assay/impurities, XRD, and SEM.

Equipment

A pilot scale reactor (R31) equipped with a mechanical stirrer and thermal circuit was used for mixing of the initial batch solutions. A pilot scale spray dryer, SD81 (Niro Mobile Minor Spray Dryer with extended chamber) was used in normal spray drying mode. A Niro 1.0 mm two-fluid atomizer was utilized and situated approximately 5 cm from the top of the spray drying vessel. An inertial cyclone separated the product from the process gas and solvent vapors. A filter bag then collected the fine particles not separated by the cyclone. The resultant gas was condensed to remove process solvents and exhausted (open cycle).

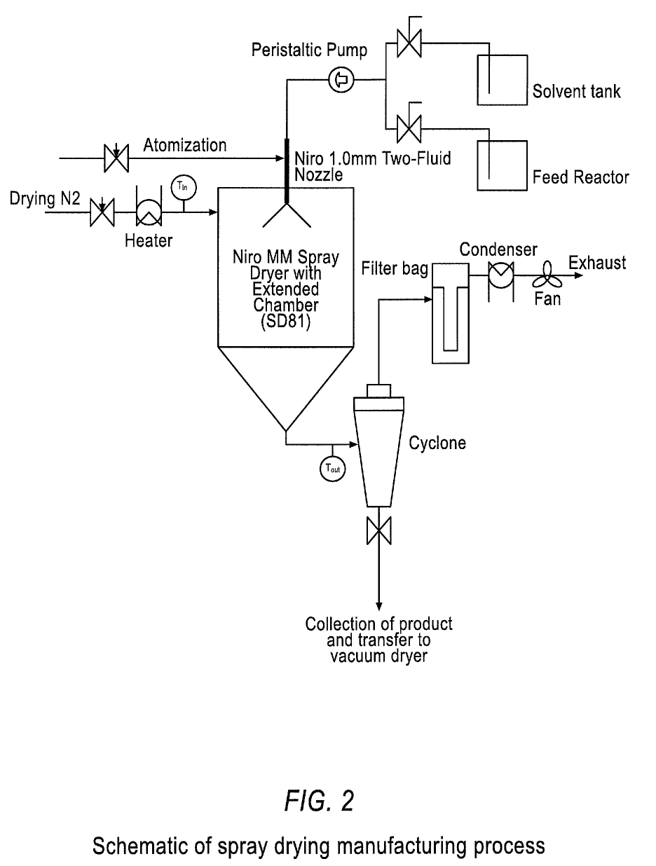

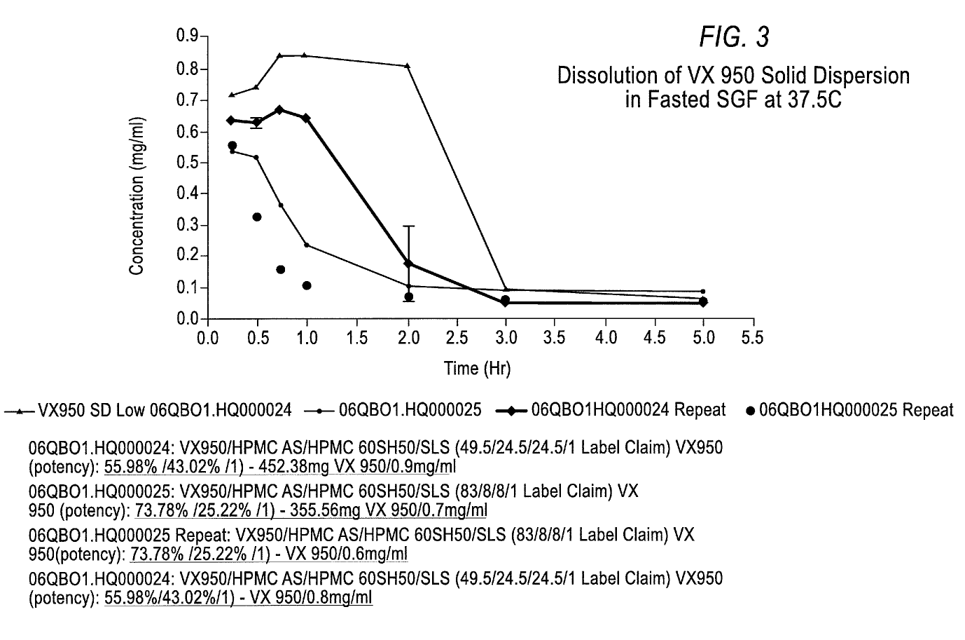

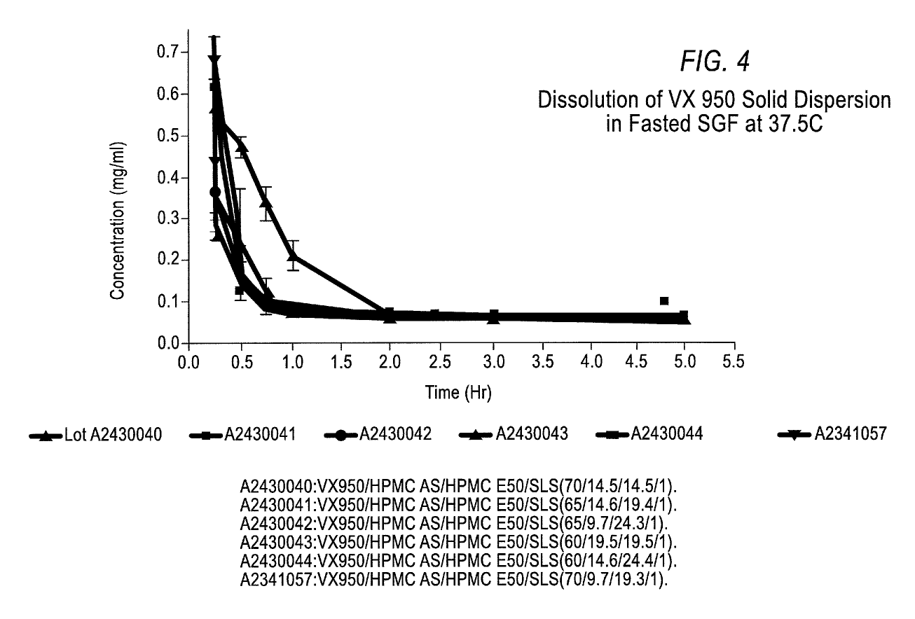

FIG. 2 is a schematic of the spray drying process.

The resultant product was transferred to a qualified vacuum dryer (EV10 or similar) for drying of residual solvents.

Key Process Control and Parameters

Key process controls and parameters were used for both the spray drying and vacuum drying process. The primary process controls parameters were identified through preliminary research batches.

Key process controls and parameters for the spray drying process, which needed to be monitored and recorded over the entirety of the run time, were:

Two-fluid Nozzle Installed

Atomization Gas Pressure and % Height of Rotameter

Inlet Temperature

Condenser Temperature Set Point (at about −15° C.)

Key process metrics for the spray drying process, which needed to be monitored and recorded over the entirety of the run time, were:

Outlet Temperature

ΔP Drying Gas

Average Solution Feed Rate for Entire Run

Table 7 defines spray drying process parameters/metrics, settings/ranges, and target guidelines.

TABLE 7
Spray drying variables, settings, and targets

| Variable | Setting/Range | |
|---|---|---|
| Two-Fluid Nozzle Installed | Niro 1.0 | mm |
| Atomization Gas Pressure | 1.5 bar and 26-40% | |
| and % Height in Rotameter | | |
| Inlet Temperature | 70-100° | C. |
| Outlet Temperature | 30-50° | C. |
| ΔP Drying Gas | 38-57 | mm H₂O |
| Average Overall Solution | 5-11 | kg/hr |
| Feedrate | | |

### Materials

All excipients and process solvents used comply with the current monographs of the European Pharmacopoeia, the Japanese Pharmacopoeia or the USP/NF as indicated herein. All excipients and process solvents were purchased from approved suppliers. Manufacturer certificate of analysis will be accepted and all materials will be ID tested.

TABLE 8
Materials

| Material | Source |
|---|---|
| Hydroxypropyl | Biddle Sawyer or Shin-Etsu |
| Methylcellulose Acetate | Chemical Co. |
| Succinate, JPE (HPMCAS) | |
| (Aqoat AS-HG) | |
| Hydroxypropyl | Biddle Sawyer or Shin-Etsu |
| Methylcellulose 60SH50 | Chemical Co. |
| (Metolose) | |
| SLS | Sigma/Fisher |
| Methylene Chloride, NF | |
| Acetone, NF | |

Additional Considerations

Dispersions can be manufactured at solid concentrations spanning from 9 wt % to 25 wt %. For example, the dispersions can be spray dried at 10 wt %.

Dispersions can be spray dried with a solvent range of 70/30 w/w methylene chloride/acetone to 100% methylene chloride. For example, the dispersions can be spray dried out of 80/20 w/w methylene chloride/acetone.

Dispersions can be spray dried with polymer(s) such as HPMCAS and/or a combination of HPMCAS-HG/HPMC. For example, the dispersions can be spray dried with the combination of HPMCAS-HG/HPMC-60SH50.

Dispersions can be spray dried with either a two-fluid nozzle or a hydraulic nozzle. For example, the dispersions can be spray dried with the two-fluid nozzle.

Handling and Storage Criteria

Upon completion of manufacture, the dispersion is packaged.

### Example 8

Solid dispersions of amorphous VX-950 comprising the ingredients given below in Table 9 were prepared (as percent weight of total dispersion) and the dissolution of the solid dispersion was measured in fasted SGF at 37.5° C. The dissolution graphs are shown in FIG. 3.

TABLE 9
Solid Dispersions of VX-950

| Dispersion | VX-950 | HPMC AS | HPMC | SLS |
|---|---|---|---|---|
| 1 | 49.5 | 24.5 | 24.5 | 1 |
| 2 | 83 | 8 | 8 | 1 |
| 3 | 83 | 8 | 8 | 1 |
| 4 | 49.5 | 24.5 | 24.5 | 1 |

### Example 9

Solid dispersions of amorphous VX-950 comprising the ingredients given below in Table 10 were prepared (as percent weight of total dispersion) and the dissolution of the solid dispersion was measured in fasted SGF at 37.5° C. The dissolution graphs are shown in FIG. 4.

TABLE 10
Solid Dispersions of VX-950

| Dispersion | VX-950 | HPMC AS | HPMC | SLS |
|---|---|---|---|---|
| 1 | 70 | 14.5 | 14.5 | 1 |
| 2 | 65 | 14.6 | 19.4 | 1 |
| 3 | 65 | 9.7 | 24.3 | 1 |
| 4 | 60 | 19.5 | 19.5 | 1 |
| 5 | 60 | 14.6 | 24.4 | 1 |
| 6 | 70 | 9.7 | 19.3 | 1 |

### Example 10

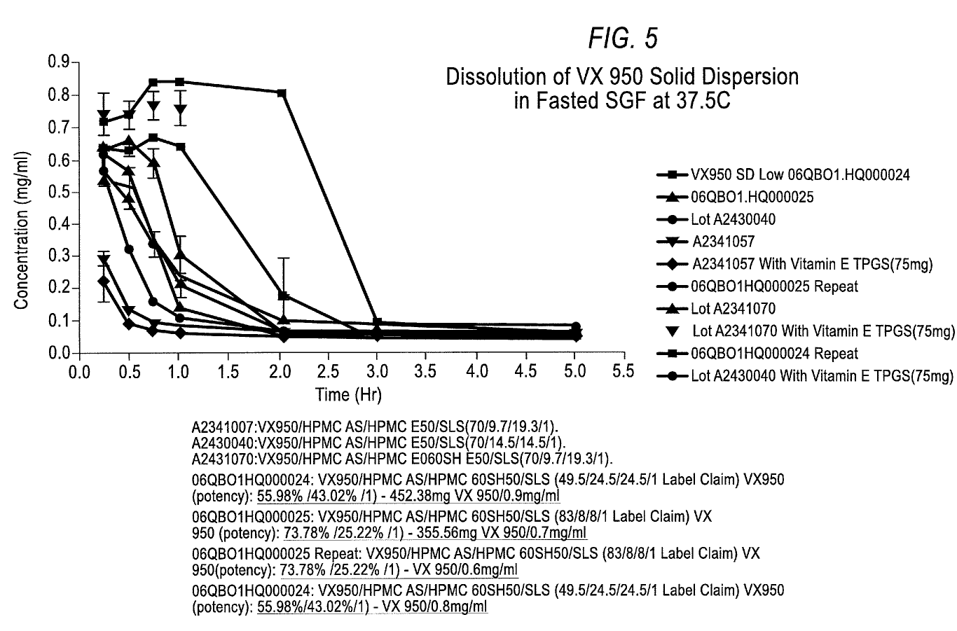

Solid dispersions of amorphous VX-950 were prepared comprising the ingredients given below in Table 11 were prepared (as percent weight of total dispersion) and the dissolution of the solid dispersion was measured in fasted SGF at 37.5° C. The dissolution graphs are shown in FIG. 5.

TABLE 11
Solid Dispersions of VX-950

| Dispersion | VX-950 | HPMC AS | HPMC | SLS |
|---|---|---|---|---|
| 1 | 70 | 9.7 | 19.3 | 1 |
| 2 | 70 | 14.5 | 14.5 | 1 |
| 3 | 70 | 9.7 | 19.3 | 1 |
| 4 | 49.5 | 24.5 | 24.5 | 1 |
| 5 | 83 | 8 | 8 | 1 |
| 6 | 83 | 8 | 8 | 1 |
| 7 | 49.5 | 24.5 | 24.5 | 1 |

### Example 11

The following solid dispersions of amorphous VX-950 were prepared with the solvent mixtures shown in Table 12. D50 and bulk density were determined for the dispersions. Values for content are given as percent weight.

TABLE 12
Solid dispersions of VX-950

| | | | Solids | | | bulk |
|---|---|---|---|---|---|---|
| ID | Formulation | Content | Conc. | Process Solvent | d50 | density |
| 13 | VX-950/HPMCAS-HG/SLS | 49.5/49.5/1 | 10 | 70/30 DCM/Acetone | 29.99 | 0.22 |
| 14 | VX-950/HPMCAS-HG/SLS | 49.5/49.5/1 | 10 | 70/30 DCM/Acetone | 20.13 | 0.25 |
| 15 | VX-950/HPMCAS-HG/SLS | 49.5/49.5/1 | 10 | 70/30 DCM/Acetone | 19.07 | 0.24 |
| 16 | VX-950/HPMCAS-HG/SLS | 49.5/49.5/1 | 10 | 70/30 DCM/Acetone | 31.9 | 0.24 |
| 17 | VX-950/HPMCAS-HG/SLS | 49.5/49.5/1 | 10 | 70/30 DCM/Acetone | 32.18 | 0.27 |
| 18 | VX-950/HPMCAS-HG/SLS | 49.5/49.5/1 | 10 | 70/30 DCM/Acetone | 20.67 | 0.26 |
| 19 | VX-950/HPMCAS-HG/SLS | 49.5/49.5/1 | 10 | 66.6/28.5/5 DCM/Acetone/GAA | 43.03 | 0.37 |
| 20 | VX-950/HPMCAS-HG/SLS | 49.5/49.5/1 | 10 | 63/27/10 DCM/Acetone/GAA | 47.02 | 0.41 |
| 21 | HPMCAS-HG | 100 | 6 | 70/30 DCM/Acetone | 38.33 | 0.24 |
| 22 | VX-950/HPMCAS-HG/SLS | 83/16/1 | 20 | 100 DCM | 33.58 | 0.33 |
| 23 | VX-950/HPMCAS-HG/SLS | ~82.44/15.89/1.67 | 20 | 100 DCM | 31.67 | 0.35 |
| 24 | VX-950/HPMCAS-HG/HPMC-60SH/SLS* | 49.5/24.75/24.75/1 | 10 | 77/23 DCM/Acetone | 22.98 | 0.31 |
| 25 | VX-950/HPMCAS-HG/HPMC-60SH/SLS* | 83/8/8/1 | 10 | 100 DCM | 27.72 | 0.35 |
| 26 | HPMCAS-HG | 100 | 4 | 100 DCM | 28.74 | 0.24 |
| 27 | VX-950/HPMCAS-HG/SLS | 83/16/1 | 10 | 100 DCM | 20.6 | 0.33 |
| A2341055 | VX-950 (amorphous) | 100 | 10 | 100 DCM | ? | ? |

*HPMC-60SH deemed equivalent to HPMCE50

### Example 12

A spray dried dispersion of amorphous VX-950 of the present disclosure can be used in preparing a tablet. The tablet can contain the formulation shown in Table 13, which contains vitamin E TPGS formulated in a melt granulate:

TABLE 13
Tableted formulation containing a
spray dried dispersion of VX-950

| | mg per | |
|---|---|---|
| Component | Tablet | Percent |
| Roller compaction blend | | |
| VX950 Spray Dried Dispersion1 | 505.1 | 74.9 |
| Pharmatose DCL 22 (Lactose, USP/NF, PhEur,) | 37.5 | 5.6 |
| Ac-Di-Sol (cross carmellose sodium, NF, PhEur, | 24.0 | 3.6 |
| Extragranular addition | | 0.0 |
| Avicel pH 113 | 33.7 | 5.0 |
| Vitamin E TPGS (NF) | 24.0 | 3.6 |
| Ac-Di-Sol (cross carmellose sodium, NF, PhEur, | 16.0 | 2.4 |
| Cabosil M-5 (colloidal silicon dioxide, NF, PhEur) | 8.0 | 1.2 |
| Sodium Stearyl fumarate (NF, PhEur, JP) | 26.0 | 3.9 |
| Total Formulation weight | 674.3 | 100.0 |

### Example 13

The examples in Table 14 are spray dried dispersions containing amorphousVX-950 that can be prepared: (Percents by weight are shown)

TABLE 14
Solid Dispersions of VX-950

| VX-950 | HPMC AS | HMPC | SLS |
|---|---|---|---|
| 60 | 24.6 | 14.4 | 1 |
| 60 | 39 | 0 | 1 |
| 49.5 | 49.5 | 0 | 1 |

A number of embodiments of the disclosure have been described. Nevertheless, it will be understood that various modifications may be made without departing from the spirit and scope of the disclosure. Accordingly, other embodiments are within the scope of the following claims.

1-33. (canceled)

34. A solid dispersion comprising about 55% VX-950, about 19.6% of an HPMC polymer, about 24.4% of an HPMCAS polymer, and about 1% of a surfactant.

35. A solid dispersion comprising about 55% VX-950, about 29.3% of an HPMC polymer, about 14.7% of an HPMCAS polymer, and about 1% of a surfactant.

36. A solid dispersion comprising about 60% VX-950, about 14.6% of an HPMC polymer, about 24.4% of an HPMCAS polymer, and about 1% of a surfactant.

37. A solid dispersion comprising about 65% VX-950, about 17% of an HPMC polymer, about 17% of an HPMCAS polymer, and about 1% of a surfactant.

38. A solid dispersion comprising about 70% VX-950, about 19.3% of an HPMC polymer, about 9.7% of an HPMCAS polymer, and about 1% of a surfactant.

39-57. (canceled)

58. A pharmaceutical composition comprising:
an amorphous solid dispersion of VX-950 comprising about 55% to about 70% wt/wt of the pharmaceutical composition,
a surfactant, wherein the surfactant is sodium lauryl sulfate or Vitamin E TPGS and comprises about 1% wt/wt of the pharmaceutical composition, and
a plurality of polymers comprises HPMC and HPMCAS, and comprises about 44% to about 29% wt/wt of the pharmaceutical composition, thereby totaling 100% wt/wt of the composition.

59. A pharmaceutical composition comprising:
an amorphous solid dispersion of VX-950 comprising about 55% wt/wt of the pharmaceutical composition,
a plurality of polymers comprising about 44% wt/wt of the pharmaceutical composition
a surfactant, wherein the surfactant is sodium lauryl sulfate or Vitamin E TPGS and comprises about 1% wt/wt of the pharmaceutical composition.

60. The pharmaceutical composition of claim 59, wherein the plurality of polymers comprises about 55.5% wt/wt HPMCAS and about 44.5% wt/wt HPMC.

61. (canceled)

62. The pharmaceutical composition of claim 59, wherein the plurality of polymers comprises about 33% wt/wt HPMCAS and about 67% wt/wt HPMC.

63. A pharmaceutical composition comprising:
an amorphous solid dispersion of VX-950 comprising about 60% wt/wt of the pharmaceutical composition,
a plurality of polymers comprising about 39% wt/wt of the pharmaceutical composition
a surfactant, wherein the surfactant is sodium lauryl sulfate or Vitamin E TPGS and comprises about 1% wt/wt of the pharmaceutical composition.

64. The pharmaceutical composition of claim 63, wherein the plurality of polymers comprises about 63% wt/wt HPMCAS and about 36% wt/wt HPMC.

65. A pharmaceutical composition comprising:
an amorphous solid dispersion of VX-950 comprising about 65% wt/wt of the pharmaceutical composition,
a plurality of polymers comprising about 34% wt/wt of the pharmaceutical composition
a surfactant, wherein the surfactant is sodium lauryl sulfate or Vitamin E TPGS and comprises about 1% wt/wt of the pharmaceutical composition.

66. The pharmaceutical composition of claim 65, wherein the plurality of polymers comprises about 50% wt/wt HPMCAS and about 50% wt/wt HPMC.

67. A pharmaceutical composition comprising:
an amorphous solid dispersion of VX-950 comprising about 70% wt/wt of the pharmaceutical composition,
a plurality of polymers comprising about 29% wt/wt of the pharmaceutical composition
a surfactant, wherein the surfactant is sodium lauryl sulfate or Vitamin E TPGS and comprises about 1% wt/wt of the pharmaceutical composition.

68. The pharmaceutical composition of claim 67, wherein the plurality of
polymers comprises about 33% wt/wt HPMCAS and about 67% wt/wt HPMC.

69-85. (canceled)
